(19) United States
(12) Patent Application Publication
(10) Pub. No.: US 2001/0008026 A1
(43) Pub. Date: Jul. 12, 2001

(54) SYSTEM FOR TISSUE-RESTRICTED GENE RECOMBINATION

(76) Inventors: MICHAEL D. SCHNEIDER, HOUSTON, TX (US); PAUL OVERBEEK, HOUSTON, TX (US); PETER FRENKEL, HOUSTON, TX (US)

Correspondence Address: Melissa L. Sistrunk, Fulbright & Jaworski L.L.P., 1301 McKinney, Suite 5100, Houston, TX, 77010-3095, US

(21) Appl. No.: 09/104,654

(22) Filed: Jun. 25, 1998

(51) Int. Cl. A01K 67/027

(52) U.S. Cl. 800/8; 800/9; 800/14; 800/18; 800/3; 800/21; 800/22; 800/25; 435/455; 435/462; 435/463; 435/465; 435/325

(57) ABSTRACT

The present invention relates to methods and compositions for tissue-restricted gene recombination. In particular, the present invention provides methods and compositions for tissue-restricted gene recombination in post-mitotic cells. The present invention further provides methods for gene recombination in post-mitotic cells comprising the delivery of a Cre recombinase to the target tissue to facilitate recombination in a desired target nucleic acid.

This invention was made in part during work partially supported by the U.S. National Institute of Health under Contracts No.: R01HL52555-02 and R01HL47567-06. The government has certain rights in the invention.

### FIELD OF THE INVENTION

The present invention relates to methods and compositions for tissue-restricted gene recombination. In particular, the present invention provides methods and compositions for tissue-restricted gene recombination in post-mitotic cells.

### BACKGROUND OF THE INVENTION

A major goal of medical genetics is to identify efficient and effective means of altering gene expression in animals (e.g., humans). In particular, methods of inhibiting, eliminating, activating, or overexpressing target genes in a tissue-specific manner are needed. Unfortunately, currently available technologies are limited in efficiency, tissue-specificity, and the ability to target differentiated, post-mitotic cells. Several of the current technologies and their limitations are discussed below.

Gene targeting to generate loss-of-function mutations using homologous recombination in the mouse has yielded remarkable advances in understanding the roles played by specific gene products in mammalian development and, to a lesser extent, human disease (Rajewsky et al., J. Clin. Invest. 98, 600 [1996]). Inherent impediments, however, can complicate the use of conventional homologous recombination to elicit the role of genes in adult pathophysiology. For example, illustrated by the retinoblastoma protein (Lee et al., Nature 359, 288 [1992]), deletion of a protein that also serves essential functions in embryogenesis can result in early lethality, precluding the use of germline mutations to investigate the protein's function in any adult context. Other potential effects confounding conventional knock-outs include the risk of impaired fertility and generalized systemic disorders, illustrated by knock-out mutations of transforming growth factor beta-1 (TGFβ1), where the ability to test specific hypotheses regarding the role of TGFβ1 in adult disease is impeded by the animals' multifocal immunological disorders, poor viability, and absolute requirement for an immunodeficient background, co-administration of immunosuppressive drugs, or both (Letterio et al., Science 264, 1936 [1994]; and Diebold et al., Proc. Natl. Acad. Sci. 92, 12215 [1995]). Related constraints are foreseeable for receptors or intracellular proteins that are required for TGFβ1 effects. Given that germline mutations thus might not permit a definitive loss-of-function study in a specific adult context, methods to achieve gene inactivation in a regulatable or tissue-restricted manner are desired.

Additionally, standard approaches for somatic cell gene transfer are not feasible in post-mitotic cells because retrovirus-mediated gene transfer requires at least one cell division for integration and expression of the gene. Thus, a critical limitation of retroviral vectors is their inability to infect non-dividing cells (Miller et al., Mol. Cell. Biol. 10:4239 [1990]). To remedy this problem, in some circumstances, cells from the target tissue can be removed, grown in vitro, and infected with the recombinant retroviral vector. The target cells containing the altered gene are then transplanted back into the animal (i.e., a procedure termed ex vivo gene therapy). However, within a few days, loss of activity from the transplanted cells is typically observed (See e.g., Dai et al., Proc. Natl. Acad. Sci. 89, 10892 [1992]), rendering this process inappropriate for many applications. Another significant problem is the possibility of random integration of vector DNA into the host chromosome. This could lead to activation of oncogenes or inactivation of tumor suppressor or other important genes. Moreover, these ex vivo approaches are inapplicable to the biological problem of deleting an endogenous gene from the majority of cells in a given tissue.

Because of the limitations and problems involved in viral transfer, methods such as direct injection of naked DNA into the non-dividing tissues have been used for gene transfer. However, the use of naked DNA is extremely inefficient for delivering genes to many post-mitotic tissues and expression of the genes transferred by such methods are largely transient (See e.g., Felgner, Sci. Am. 276:102 [1997]).

In view of the aforementioned insufficiencies associated with prior art methods of gene targeting, it is apparent that there exists a need for a gene targeting method that provides efficient gene deletion in a variety of cell types, including post-mitotic cells.

### SUMMARY OF THE INVENTION

The present invention relates to methods and compositions for tissue-restricted gene recombination. In particular, the present invention provides methods and compositions for tissue-restricted gene recombination in post-mitotic cells.

The present invention provides a method for gene recombination in post-mitotic cells, comprising: providing a gene transfer system comprising a DNA sequence encoding a Cre recombinase and post-mitotic target tissue comprising target nucleic acid, said target nucleic acid comprising one or more site-specific recombination target sequences; and introducing the gene transfer system to the target tissue, whereby recombination occurs at the one or more site-specific recombination target sequences. In one embodiment of the present invention, the DNA sequence encoding a Cre recombinase further comprises a tissue-specific promoter sequence.

In preferred embodiments, the gene transfer system comprises a viral gene transfer system. In particularly preferred embodiments, the viral gene transfer system comprises an adenoviral gene transfer system.

In certain embodiments of the present invention, the post-mitotic target tissue comprises cardiac tissue, although the methods of the present invention are applicable to any post-mitotic target tissue. In other embodiments, the target nucleic acid comprises a gene. In yet other embodiments, the one or more site-specific recombination target sequences comprises one or more loxP target sequences, although other site-specific recombination target sequences are contemplated by the present invention, including, but not limited to, loxP2, loxP3, loxP23, loxP511, loxB, loxC2, loxL, loxR, loxΔ86, loxΔ117, frt, dif, flp, and att target sequences.

In certain embodiments of the present invention, the introducing of the gene transfer system to the target tissue, comprises injecting the gene transfer system into the target tissue.

The present invention further provides a method for gene recombination in cardiac tissue, comprising: providing a viral gene transfer system comprising a DNA sequence encoding a Cre recombinase and cardiac tissue comprising target nucleic acid, said target nucleic acid comprising one or more site-specific recombination target sequences; and introducing the viral gene transfer system to the cardiac tissue, whereby recombination occurs at the one or more site-specific recombination target sequences. In some embodiments of the present invention, the cardiac tissue comprises post-mitotic cardiac tissue. In other embodiments, the DNA sequence encoding a Cre recombinase further comprises a cardiac-specific promoter sequence. In yet other embodiments, the introducing of the viral gene transfer system to the cardiac tissue, comprises injecting the viral gene transfer system into the cardiac tissue.

The present invention further provides a method for cardiac-restricted gene recombination, comprising: providing a viral gene transfer system comprising a DNA sequence encoding a Cre recombinase and a cardiac-specific promoter sequence and post-mitotic cardiac tissue comprising target nucleic acid, said target nucleic acid comprising one or more site-specific recombination target sequences; and introducing the viral gene transfer system to the post-mitotic cardiac tissue, whereby recombination occurs at the one or more site-specific recombination target sequences. In some embodiments, the introducing of the viral gene transfer system to the post-mitotic cardiac tissue comprises injecting the viral gene transfer system into the post-mitotic cardiac tissue. In certain embodiments, the cardiac-specific promoter sequence comprises an α-myosin heavy chain promoter sequence.

The present invention further provides a non-human mammal, wherein one or more tissues of the non-human mammal comprise tissue prepared according to the methods described above. In preferred embodiments, the non-human mammal is selected from the order Rodentia. In particularly preferred embodiments, the non-human mammal is selected from the group consisting of mice and rats.

The present invention further provides a non-human mammal having post-mitotic tissue comprising an altered genotype as compared to wild-type post-mitotic tissue, wherein the altered genotype is the result of tissue-specific recombination. In preferred embodiments the post-mitotic tissue comprises cardiac tissue, although the present invention contemplates all other post-mitotic tissues. In some embodiments, the altered genotype comprises a gene knockout.

The present invention further provides a method for screening compounds for their effect on a transgenic animal, comprising: providing a transgenic animal, wherein the transgenic animal is the non-human mammal described above and a composition comprising a test compound in a form suitable for administration to the non-human mammal; and administering the test compound to the non-human mammal. In some embodiments, the method further comprises the step of detecting a response of the non-human mammal to the test compound.

### DESCRIPTION OF THE FIGURES

FIG. 1 shows a schematic of pCAG-CATZ. The PCR primers, AG and Z3, flank the paired loxP sites and internal CAT gene.

FIG. 2 shows relative LacZ from αMyHC-driven Cre vectors mediating recombination in vitro in A) myocytes or B) fibroblasts; and C) relative CAT activity in fibroblasts.

FIG. 3 shows PCR analysis of αMyHC-driven Cre vector-mediated recombination in cardiac muscle cells.

FIG. 4 shows the structure of a αMyHC-Cre construct.

FIG. 5 shows PCR analysis of representative littermates from the mating of an αMyHC-Cre⁺mouse to a CAG-CATZ⁺mouse.

FIG. 6 shows whole-organ staining in littermates resulting from an αMyHC-Cre/CAG-CATZ cross.

FIG. 7 shows histochemical staining for LacZ expression in mycocardium of transgenic mice.

FIG. 8 shows X-gal staining of tissues following adenoviral gene transfer to adult mouse myocardium in vivo where the panels represent A) vehicle injection (500 μm); B) vehicle injection (100 μm); C) Ad5/CMV/nls-lacZ (500 μm); D) Ad5/CMV/nls-lacZ (100 μm); E) Ad5/CMV (500 μm); F) Ad5/CMV (100 μm); and G) the proportion of LacZ-positive cells relative to the total cell number from hematoxylin-eosin-stained sections treated with the vehicle, Ad5/CMV/nls-lacZ, or Ad5/CMV.

FIG. 9 shows a schematic representation of AdMA19. The spacer interposed between the loxP sites precludes efficient luciferase expression in the absence of the Cre recombinase.

FIG. 10 shows luciferase induction in neonatal rat cardiac myocytes with AdMA19, with and without coinfection with AdCre.

FIG. 11 shows a Western blot analysis of Cre-dependent luciferase expression.

FIG. 12 shows luciferase activity in adult mouse myocardium following injection with adenoviruses bearing CMV-Cre or MA19 singly or in combination.

FIG. 13 shows luciferase activity detected by immunoperoxidase staining of ventricular myocytes using the indicated viruses and antibodies.

FIG. 14 shows X-gal staining of cryostat sections of adult mouse myocardium injected with adenovirus bearing CMV-Cre (A-D) or empty CMV control (E-H).

FIG. 15 shows A) the specific deletion of exon 19 from the retinoblastoma (Rb) gene in cardiac muscle (H) (as opposed to kidney [K], lung [L], or liver [Li]), when αMyHC-Cre mice are mated to mice with a loxP-tagged Rb gene; and B) the resulting increase in DNA synthesis in heart muscle as shown by immunostaining for the DNA precursor 5′-bromodeoxyuridine.

### DEFINITIONS

To facilitate an understanding of the invention, a number of terms are defined below.

As used herein, the term “tissue-restricted recombination” refers to recombination that occurs in specific tissues or subsets of tissues. Tissue-restricted recombination can occur, for example, because of the presence of an active recombinase enzyme in particular tissues but not others. “Cardiac-restricted recombination” refers to recombination that occurs specifically in cardiac tissue and not other tissues.

As used herein, the term “post-mitotic cells” refers to quiescent cells (i.e., cells that are not, or are not capable of, undergoing cell division). Many fully differentiated cells are post-mitotic cells. As used herein, “post-mitotic target tissue” refers to tissue comprising post-mitotic cells. Such tissues may also be associated with mitotic cells (i.e., cells that are dividing or capable of dividing). As used herein, the term “post-mitotic cardiac tissue” refers to heart tissue that comprises post-mitotic cells (e.g., cardiac myocytes). Post-mitotic cardiac tissue may also be associated with mitotic cells (e.g., cardiac fibroblasts). The methods of the present invention provide means for recombination in post-mitotic cells in post-mitotic tissues. In contrast to the present invention, prior methods do not demonstrate Cre-driven recombination in post-mitotic cells (e.g., the recombination of the prior methods occurred in the mitotic cells of the tissues [e.g., fibroblasts] as opposed to post-mitotic cells [e.g., myocytes]).

As used herein, the term “gene targeting” refers to the alteration of genes through molecular biology techniques. Such gene targeting includes, but is not limited to, generation of mutant genes and knockout genes through recombination. When a gene is altered such that its product is no longer biologically active in a wild-type fashion, the mutation is referred to as a “loss-of-function” mutation. When a gene is altered such that a portion or the entirety of the gene is deleted or replaced, the mutation is referred to as a “knockout” mutation.

As used herein, the term “gene transfer system” refers to any means of delivering a composition comprising a nucleic acid sequence to a cell or tissue. For example, gene transfer systems include, but are not limited to vectors (e.g., retroviral, adenoviral, adeno-associated viral, and other nucleic acid-based delivery systems), microinjection of naked nucleic acid, and polymer-based delivery systems (e.g., liposome-based and metallic particle-based systems). As used herein, the term “viral gene transfer system” refers to gene transfer systems comprising viral elements (e.g., intact viruses and modified viruses) to facilitate delivery of the sample to a desired cell or tissue. As used herein, the term “adenovirus gene transfer system” refers to gene transfer systems comprising intact or altered viruses belonging to the family Adenoviridae.

As used herein, the term “site-specific recombination target sequences” refers to nucleic acid sequences that provide recognition sequences for recombination factors and the location where recombination takes place.

The term “biologically active,” as used herein, refers to a protein or other biologically active molecules (e.g., catalytic RNA) having structural, regulatory, or biochemical functions of a naturally occurring molecule.

The term “agonist,” as used herein, refers to a molecule which, when interacting with an biologically active molecule, causes a change (e.g., enhancement) in the biologically active molecule, which modulates the activity of the biologically active molecule. Agonists may include proteins, nucleic acids, carbohydrates, or any other molecules which bind or interact with biologically active molecules. For example, agonists can alter the activity of gene transcription by interacting with RNA polymerase directly or through a transcription factor.

The terms “antagonist” or “inhibitor,” as used herein, refer to a molecule which, when interacting with a biologically active molecule, blocks or modulates the biological activity of the biologically active molecule. Antagonists and inhibitors may include proteins, nucleic acids, carbohydrates, or any other molecules that bind or interact with biologically active molecules. Inhibitors and antagonists can effect the biology of entire cells, organs, or organisms (e.g., an inhibitor that slows tumor growth).

The term “modulate,” as used herein, refers to a change in the biological activity of a biologically active molecule. Modulation can be an increase or a decrease in activity, a change in binding characteristics, or any other change in the biological, functional, or immunological properties of biologically active molecules.

As used herein, the term “nucleic acid molecule” refers to any nucleic acid containing molecule including, but not limited to DNA or RNA. The term encompasses sequences that include any of the known base analogs of DNA and RNA including, but not limited to, 4-acetylcytosine, 8-hydroxy-N6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

The term “gene” refers to a nucleic acid (e.g., DNA) sequence that comprises coding sequences necessary for the production of a polypeptide or precursor (e.g., α-myosin heavy chain). The polypeptide can be encoded by a full length coding sequence or by any portion of the coding sequence so long as the desired activity or functional properties (e.g., enzymatic activity, ligand binding, signal transduction, etc.) of the full-length coding sequence or fragment of the full-length coding sequence are retained. The term also encompasses the coding region of a structural gene and the including sequences located adjacent to the coding region on both the 5′and 3′ends for a distance of about 1 kb or more on either end such that the gene corresponds to the length of the full-length mRNA. The sequences that are located 5′of the coding region and which are present on the mRNA are referred to as 5′non-translated sequences. The sequences that are located 3′or downstream of the coding region and which are present on the mRNA are referred to as 3′non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene which are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “gene expression” refers to the process of converting genetic information encoded in a gene into RNA (e.g., mRNA, rRNA, tRNA, or snRNA) through “transcription” of the gene (i.e., via the enzymatic action of an RNA polymerase), and for protein encoding genes, into protein through “translation” of mRNA. Gene expression can be regulated at many stages in the process. “Up-regulation” or “activation” refers to regulation that increases the production of gene expression products (i.e., RNA or protein), while “down-regulation” or “repression” refers to regulation that decrease production. Molecules (e.g., transcription factors) that are involved in up-regulation or down-regulation are often called “activators” and “repressors,” respectively.

Where “amino acid sequence” is recited herein to refer to an amino acid sequence of a naturally occurring protein molecule, “amino acid sequence” and like terms, such as “polypeptide” or “protein” are not meant to limit the amino acid sequence to the complete, native amino acid sequence associated with the recited protein molecule.

In addition to containing introns, genomic forms of a gene may also include sequences located on both the 5′and 3′end of the sequences which are present on the RNA transcript. These sequences are referred to as “flanking” sequences or regions (these flanking sequences are located 5′or 3′to the non-translated sequences present on the mRNA transcript). The 5′flanking region may contain regulatory sequences such as promoters and enhancers that control or influence the transcription of the gene. The 3′flanking region may contain sequences which direct the termination of transcription, post-transcriptional cleavage and polyadenylation.

The term “wild-type” refers to a gene or gene product which has the characteristics of that gene or gene product when isolated from a naturally occurring source. A wild-type gene is that which is most frequently observed in a population and is thus arbitrarily designed the “normal” or “wild-type” form of the gene. In contrast, the terms “modified,” “mutant,” and “altered genotype” refer to a gene or gene product which displays modifications in sequence and/or functional properties when compared to the wild-type gene or gene product. It is noted that naturally-occurring mutants can be isolated; these are identified by the fact that they have altered characteristics when compared to the wild-type gene or gene product.

As used herein, the terms “nucleic acid molecule encoding,” “DNA sequence encoding,” and “DNA encoding” refer to the order or sequence of deoxyribonucleotides along a strand of deoxyribonucleic acid that encodes a particular protein or amino acid sequence. The order of these deoxyribonucleotides determines the order of amino acids along the polypeptide (protein) chain.

DNA molecules are said to have “5′ends” and “3′ends” because mononucleotides are reacted to make oligonucleotides or polynucleotides in a manner such that the 5′phosphate of one mononucleotide pentose ring is attached to the 3′ oxygen of its neighbor in one direction via a phosphodiester linkage. Therefore, an end of an oligonucleotides or polynucleotide, referred to as the “5′end” if its 5′ phosphate is not linked to the 3′oxygen of a mononucleotide pentose ring and as the “3′end” if its 3′oxygen is not linked to a 5′phosphate of a subsequent mononucleotide pentose ring. As used herein, a nucleic acid sequence, even if internal to a larger oligonucleotide or polynucleotide, also may be said to have 5′and 3′ends. In either a linear or circular DNA molecule, discrete elements are referred to as being “upstream” or 5′of the “downstream” or 3′elements. This terminology reflects the fact that transcription proceeds in a 5′to 3′fashion along the DNA strand. The promoter and enhancer elements that direct transcription of a linked gene are generally located 5′or upstream of the coding region. However, enhancer elements can exert their effect even when located 3′of the promoter element or the coding region. Transcription termination and polyadenylation signals are located 3′or downstream of the coding region.

As used herein, the terms “an oligonucleotide having a nucleotide sequence encoding a gene” and “polynucleotide having a nucleotide sequence encoding a gene,” means a nucleic acid sequence comprising the coding region of a gene or in other words the nucleic acid sequence which encodes a gene product. The coding region may be present in either a cDNA, genomic DNA or RNA form. When present in a DNA form, the oligonucleotide or polynucleotide may be single-stranded (i.e., the sense strand) or double-stranded. Suitable control elements such as enhancers/promoters, splice junctions, polyadenylation signals, etc. may be placed in close proximity to the coding region of the gene if needed or desired, to permit proper initiation of transcription and/or correct processing of the primary RNA transcript. Alternatively, the coding region utilized in the expression vectors of the present invention may contain endogenous enhancers/promoters, splice junctions, intervening sequences, polyadenylation signals, etc. or a combination of both endogenous and exogenous control elements.

As used herein, the term “oligonucleotide,” refers to a short length of single-stranded polynucleotide chain. Oligonucleotides are typically less than 100 residues long (e.g., between 15 and 50), however, as used herein, the term is also intended to encompass longer polynucleotide chains. Oligonucleotides are often referred to by their length. For example a 24 residue oligonucleotide is referred to as a “24-mer”. Oligonucleotides can form secondary and tertiary structures by self-hybridizing or by hybridizing to other polynucleotides. Such structures can include, but are not limited to, duplexes, hairpins, cruciforms, bends, and triplexes.

As used herein, the term “regulatory element” refers to a genetic element which controls some aspect of the expression of nucleic acid sequences. For example, a promoter is a regulatory element that facilitates the initiation of transcription of an operably linked coding region. Other regulatory elements are splicing signals, polyadenylation signals, termination signals, etc. (defined infra).

Transcriptional control signals in eukaryotes comprise “promoter” and “enhancer” elements. Promoters and enhancers consist of short arrays of DNA sequences that interact specifically with cellular proteins involved in transcription (T. Maniatis et al., Science 236:1237 [1987]). Promoter and enhancer elements have been isolated from a variety of eukaryotic sources including genes in yeast, insect and mammalian cells, and viruses (analogous control elements, i.e., promoters, are also found in prokaryotes). The selection of a particular promoter and enhancer depends on what cell type is to be used to express the protein of interest. Some eukaryotic promoters and enhancers have a broad host range while others are functional in a limited subset of cell types (for review see, S. D. Voss et al., Trends Biochem. Sci., 11:287 [1986]; and T. Maniatis et al., supra). For example, the SV40 early gene enhancer is very active in a wide variety of cell types from many mammalian species and has been widely used for the expression of proteins in mammalian cells (R. Dijkema et al., EMBO J. 4:761 [1985]). Two other examples of promoter/enhancer elements active in a broad range of mammalian cell types are those from the human elongation factor 1α gene (T. Uetsuki et al., J. Biol. Chem., 264:5791 [1989]; D. W. Kim et al., Gene 91:217 [1990]; and S. Mizushima and S. Nagata, Nuc. Acids. Res., 18:5322 [1990]) and the long terminal repeats of the Rous sarcoma virus (C. M. Gorman et al., Proc. Natl. Acad. Sci. USA 79:6777 [1982]) and the human cytomegalovirus (M. Boshart et al., Cell 41:521 [1985]). Some promoter elements serve to direct gene expression in a tissue-specific manner. For example, the murine α-myosin-heavy chain promoter, α-5.5, promotes expression in a cardiac-specific manner.

As used herein, the term “promoter/enhancer” denotes a segment of DNA which contains sequences capable of providing both promoter and enhancer functions (i.e., the functions provided by a promoter element and an enhancer element, see above for a discussion of these functions). For example, the long terminal repeats of retroviruses contain both promoter and enhancer functions. The enhancer/promoter may be “endogenous” or “exogenous” or “heterologous.” An “endogenous” enhancer/promoter is one which is naturally linked with a given gene in the genome. An “exogenous” or “heterologous” enhancer/promoter is one which is placed in juxtaposition to a gene by means of genetic manipulation (i.e., molecular biological techniques such as cloning and recombination) such that transcription of that gene is directed by the linked enhancer/promoter.

The presence of “splicing signals” on an expression vector often results in higher levels of expression of the recombinant transcript. Splicing signals mediate the removal of introns from the primary RNA transcript and consist of a splice donor and acceptor site (J. Sambrook et al., Molecular Cloning: A Laboratory Manual, 2nd ed., Cold Spring Harbor Laboratory Press, New York [1989], pp. 16.7-16.8). A commonly used splice donor and acceptor site is the splice junction from the 16S RNA of SV40.

Efficient expression of recombinant DNA sequences in eukaryotic cells requires expression of signals directing the efficient termination and polyadenylation of the resulting transcript. Transcription termination signals are generally found downstream of the polyadenylation signal and are a few hundred nucleotides in length. The term “poly A site” or “poly A sequence” as used herein denotes a DNA sequence that directs both the termination and polyadenylation of the nascent RNA transcript. Efficient polyadenylation of the recombinant transcript is desirable as transcripts lacking a poly A tail are unstable and are rapidly degraded. The poly A signal utilized in an expression vector may be “heterologous” or “endogenous.” An endogenous poly A signal is one that is found naturally at the 3′end of the coding region of a given gene in the genome. A heterologous poly A signal is one that is isolated from one gene and placed 3′of another gene. A commonly used heterologous poly A signal is the SV40 poly A signal. The SV40 poly A signal is contained on a 237 bp BamHI/BclI restriction fragment and directs both termination and polyadenylation (J. Sambrook, supra, at 16.6-16.7).

Eukaryotic expression vectors may also contain “viral replicons” or “viral origins of replication.” Viral replicons are viral DNA sequences that allow for the extrachromosomal replication of a vector in a host cell expressing the appropriate replication factors. Vectors that contain either the SV40 or polyoma virus origin of replication replicate to high “copy number” (up to 10⁴ copies/cell) in cells that express the appropriate viral T antigen. Vectors that contain the replicons from bovine papillomavirus or Epstein-Barr virus replicate extrachromosomally at “low copy number” (˜100 copies/cell).

As used herein, the terms “complementary” or “complementarity” are used in reference to polynucleotides (i.e., a sequence of nucleotides) related by the base-pairing rules. For example, for the sequence “A-G-T,” is complementary to the sequence “T-C-A.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

The term “homology” refers to a degree of complementarity. There may be partial homology or complete homology (i.e., identity). A partially complementary sequence is one that at least partially inhibits a completely complementary sequence from hybridizing to a target nucleic acid is referred to using the functional term “substantially homologous.” The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous to a target under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target that lacks even a partial degree of complementarity (e.g., less than about 30% identity); in the absence of non-specific binding the probe will not hybridize to the second non-complementary target.

The art knows well that numerous equivalent conditions may be employed to comprise low stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol) are considered and the hybridization solution may be varied to generate conditions of low stringency hybridization different from, but equivalent to, the above listed conditions. In addition, the art knows conditions that promote hybridization under conditions of high stringency (e.g., increasing the temperature of the hybridization and/or wash steps, the use of formamide in the hybridization solution, etc.) (see definition below for “stringency”).

When used in reference to a double-stranded nucleic acid sequence such as a cDNA or genomic clone, the term “substantially homologous” refers to any probe that can hybridize to either or both strands of the double-stranded nucleic acid sequence under conditions of low stringency as described above.

A gene may produce multiple RNA species that are generated by differential splicing of the primary RNA transcript. cDNAs that are splice variants of the same gene will contain regions of sequence identity or complete homology (representing the presence of the same exon or portion of the same exon on both cDNAs) and regions of complete non-identity (for example, representing the presence of exon “A” on cDNA 1 wherein cDNA 2 contains exon “B” instead). Because the two cDNAs contain regions of sequence identity they will both hybridize to a probe derived from the entire gene or portions of the gene containing sequences found on both cDNAs; the two splice variants are therefore substantially homologous to such a probe and to each other.

When used in reference to a single-stranded nucleic acid sequence, the term “substantially homologous” refers to any probe that can hybridize (i.e., it is the complement of) the single-stranded nucleic acid sequence under conditions of low stringency as described above.

As used herein, the term “hybridization” is used in reference to the pairing of complementary nucleic acids. Hybridization and the strength of hybridization (i.e., the strength of the association between the nucleic acids) is impacted by such factors as the degree of complementary between the nucleic acids, stringency of the conditions involved, the Tₘ of the formed hybrid, and the G:C ratio within the nucleic acids. A single molecule that contains pairing of complementary nucleic acids within its structure is said to be “self-hybridized.”

As used herein, the term “Tₘ” is used in reference to the “melting temperature.” The melting temperature is the temperature at which a population of double-stranded nucleic acid molecules becomes half dissociated into single strands. The equation for calculating the Tₘ of nucleic acids is well known in the art. As indicated by standard references, a simple estimate of the Tₘ value may be calculated by the equation: Tₘ=81.5+0.41(% G+C), when a nucleic acid is in aqueous solution at 1 M NaCl (See e.g., Anderson and Young, Quantitative Filter Hybridization, in Nucleic Acid Hybridization [1985]). Other references include more sophisticated computations that take structural as well as sequence characteristics into account for the calculation of Tₘ.

As used herein the term “stringency” is used in reference to the conditions of temperature, ionic strength, and the presence of other compounds such as organic solvents, under which nucleic acid hybridizations are conducted. With “high stringency” conditions, nucleic acid base pairing will occur only between nucleic acid fragments that have a high frequency of complementary base sequences. Thus, conditions of “weak” or “low” stringency are often required with nucleic acids that are derived from organisms that are genetically diverse, as the frequency of complementary sequences is usually less.

“Amplification” is a special case of nucleic acid replication involving template specificity. It is to be contrasted with non-specific template replication (i.e., replication that is template-dependent but not dependent on a specific template). Template specificity is here distinguished from fidelity of replication (i.e., synthesis of the proper polynucleotide sequence) and nucleotide (ribo- or deoxyribo-) specificity. Template specificity is frequently described in terms of “target” specificity. Target sequences are “targets” in the sense that they are sought to be sorted out from other nucleic acid. Amplification techniques have been designed primarily for this sorting out.

Template specificity is achieved in most amplification techniques by the choice of enzyme. Amplification enzymes are enzymes that, under conditions they are used, will process only specific sequences of nucleic acid in a heterogeneous mixture of nucleic acid. For example, in the case of Qβ replicase, MDV-1 RNA is the specific template for the replicase (D. L. Kacian et al., Proc. Natl. Acad. Sci. USA 69:3038 [1972]). Other nucleic acid will not be replicated by this amplification enzyme. Similarly, in the case of T7 RNA polymerase, this amplification enzyme has a stringent specificity for its own promoters (M. Chamberlin et al., Nature 228:227 [1970]). In the case of T4 DNA ligase, the enzyme will not ligate the two oligonucleotides or polynucleotides, where there is a mismatch between the oligonucleotide or polynucleotide substrate and the template at the ligation junction (D. Y. Wu and R. B. Wallace, Genomics 4:560 [1989]). Finally, Taq and Pfu polymerases, by virtue of their ability to function at high temperature, are found to display high specificity for the sequences bounded and thus defined by the primers; the high temperature results in thermodynamic conditions that favor primer hybridization with the target sequences and not hybridization with non-target sequences (H. A. Erlich (ed.), PCR Technology, Stockton Press [1989]).

As used herein, the term “amplifiable nucleic acid” is used in reference to nucleic acids which may be amplified by any amplification method. It is contemplated that “amplifiable nucleic acid” will usually comprise “sample template.”

As used herein, the term “sample template” refers to nucleic acid originating from a sample that is analyzed for the presence of “target.” In contrast, “background template” is used in reference to nucleic acid other than sample template which may or may not be present in a sample. Background template is most often inadvertent. It may be the result of carryover, or it may be due to the presence of nucleic acid contaminants sought to be purified away from the sample. For example, nucleic acids from organisms other than those to be detected may be present as background in a test sample.

As used herein, the term “primer” refers to an oligonucleotide, whether occurring naturally as in a purified restriction digest or produced synthetically, that is capable of acting as a point of initiation of synthesis when placed under conditions in which synthesis of a primer extension product that is complementary to a nucleic acid strand is induced, (i.e., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer and the use of the method.

As used herein, the term “probe” refers to an oligonucleotide (ie., a sequence of nucleotides), whether occurring naturally as in a purified restriction digest or produced synthetically, recombinantly or by PCR amplification, that is capable of hybridizing to another oligonucleotide of interest. A probe may be single-stranded or double-stranded. Probes are useful in the detection, identification and isolation of particular gene sequences. It is contemplated that any probe used in the present invention will be labelled with any “reporter molecule,” so that is detectable in any detection system, including, but not limited to enzyme (e.g., ELISA, as well as enzyme-based histochemical assays), fluorescent, radioactive, and luminescent systems. It is not intended that the present invention be limited to any particular detection system or label.

As used herein, the term “target,” refers to the region of nucleic acid bounded by the primers. Thus, the “target” is sought to be sorted out from other nucleic acid sequences. A “segment” is defined as a region of nucleic acid within the target sequence.

As used herein, the term “polymerase chain reaction” (“PCR”) refers to the method of K. B. Mullis U.S. Pat. Nos. 4,683,195 4,683,202, and 4,965,188, hereby incorporated by reference, which describe a method for increasing the concentration of a segment of a target sequence in a mixture of genomic DNA without cloning or purification. This process for amplifying the target sequence consists of introducing a large excess of two oligonucleotide primers to the DNA mixture containing the desired target sequence, followed by a precise sequence of thermal cycling in the presence of a DNA polymerase. The two primers are complementary to their respective strands of the double stranded target sequence. To effect amplification, the mixture is denatured and the primers then annealed to their complementary sequences within the target molecule. Following annealing, the primers are extended with a polymerase so as to form a new pair of complementary strands. The steps of denaturation, primer annealing and polymerase extension can be repeated many times (i.e., denaturation, annealing and extension constitute one “cycle”; there can be numerous “cycles”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified.”

With PCR, it is possible to amplify a single copy of a specific target sequence in genomic DNA to a level detectable by several different methodologies (e.g., hybridization with a labeled probe; incorporation of biotinylated primers followed by avidin-enzyme conjugate detection; incorporation of ³²P-labeled deoxynucleotide triphosphates, such as dCTP or dATP, into the amplified segment). In addition to genomic DNA, any oligonucleotide or polynucleotide sequence can be amplified with the appropriate set of primer molecules. In particular, the amplified segments created by the PCR process are, themselves, efficient templates for subsequent PCR amplifications.

As used herein, the terms “PCR product,” “PCR fragment,” and “amplification product” refer to the resultant mixture of compounds after two or more cycles of the PCR steps of denaturation, annealing and extension are complete. These terms encompass the case where there has been amplification of one or more segments of one or more target sequences.

As used herein, the term “amplification reagents” refers to those reagents (deoxyribonucleotide triphosphates, buffer, etc.), needed for amplification except for primers, nucleic acid template and the amplification enzyme. Typically, amplification reagents along with other reaction components are placed and contained in a reaction vessel (test tube, microwell, etc.).

As used herein, the terms “restriction endonucleases” and “restriction enzymes” refer to bacterial enzymes, each of which cut double-stranded DNA at or near a specific nucleotide sequence.

As used herein, the term “antisense” is used in reference to DNA or RNA sequences that are complementary to a specific DNA or RNA sequence (e.g., mRNA). Included within this definition are antisense RNA (“asRNA”) molecules involved in gene regulation by bacteria. Antisense RNA may be produced by any method, including synthesis by splicing the gene(s) of interest in a reverse orientation to a viral promoter which permits the synthesis of a coding strand. Once introduced into an embryo, this transcribed strand combines with natural mRNA produced by the embryo to form duplexes. These duplexes then block either the further transcription of the mRNA or its translation. In this manner, mutant phenotypes may be generated. The term “antisense strand” is used in reference to a nucleic acid strand that is complementary to the “sense” strand. The designation (−) (i.e., “negative”) is sometimes used in reference to the antisense strand, with the designation (+) sometimes used in reference to the sense (ie., “positive”) strand.

The terms “in operable combination,” “in operable order,” and “operably linked” as used herein refer to the linkage of nucleic acid sequences in such a manner that a nucleic acid molecule capable of directing the transcription of a given gene and/or the synthesis of a desired protein molecule is produced. The term also refers to the linkage of amino acid sequences in such a manner so that a functional protein is produced.

The term “isolated” when used in relation to a nucleic acid, as in “an isolated oligonucleotide” or “isolated polynucleotide” refers to a nucleic acid sequence that is identified and separated from at least one contaminant nucleic acid with which it is ordinarily associated in its natural source. Isolated nucleic acid is such present in a form or setting that is different from that in which it is found in nature. In contrast, non-isolated nucleic acids as nucleic acids such as DNA and RNA found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. However, isolated nucleic acid encoding a given protein includes, by way of example, such nucleic acid in cells ordinarily expressing the given protein where the nucleic acid is in a chromosomal location different from that of natural cells, or is otherwise flanked by a different nucleic acid sequence than that found in nature. The isolated nucleic acid, oligonucleotide, or polynucleotide may be present in single-stranded or double-stranded form. When an isolated nucleic acid, oligonucleotide or polynucleotide is to be utilized to express a protein, the oligonucleotide or polynucleotide will contain at a minimum the sense or coding strand (i.e., the oligonucleotide or polynucleotide may be single-stranded), but may contain both the sense and anti-sense strands (i.e., the oligonucleotide or polynucleotide may be double-stranded).

As used herein, the term “purified” or “to purify” refers to the removal of contaminants from a sample. For example, antibodies are purified by removal of contaminating non-immunoglobulin proteins; they are also purified by the removal of immunoglobulin that does not bind to the target molecule. The removal of non-immunoglobulin proteins and/or the removal of immunoglobulins that do not bind to the target molecule results in an increase in the percent of target-reactive immunoglobulins in the sample. In another example, recombinant polypeptides are expressed in bacterial host cells and the polypeptides are purified by the removal of host cell proteins; the percent of recombinant polypeptides is thereby increased in the sample.

The term “recombinant DNA molecule” as used herein refers to a DNA molecule that is comprised of segments of DNA joined together by means of molecular biological techniques.

The term “recombinant protein” or “recombinant polypeptide” as used herein refers to a protein molecule that is expressed from a recombinant DNA molecule.

The term “native protein” as used herein to indicate that a protein does not contain amino acid residues encoded by vector sequences; that is the native protein contains only those amino acids found in the protein as it occurs in nature. A native protein may be produced by recombinant means or may be isolated from a naturally occurring source.

As used herein the term “portion” when in reference to a protein (as in “a portion of a given protein”) refers to fragments of that protein. The fragments may range in size from four amino acid residues to the entire amino acid sequence minus one amino acid.

The term “Southern blot,” refers to the analysis of DNA on agarose or acrylamide gels to fractionate the DNA according to size followed by transfer of the DNA from the gel to a solid support, such as nitrocellulose or a nylon membrane. The immobilized DNA is then probed with a labeled probe to detect DNA species complementary to the probe used. The DNA may be cleaved with restriction enzymes prior to electrophoresis. Following electrophoresis, the DNA may be partially depurinated and denatured prior to or during transfer to the solid support. Southern blots are a standard tool of molecular biologists (J. Sambrook et al., Molecular Cloning: A Laboratory Manual, Cold Spring Harbor Press, NY, pp 9.31-9.58 [1989]).

The term “Northern blot,” as used herein refers to the analysis of RNA by electrophoresis of RNA on agarose gels to fractionate the RNA according to size followed by transfer of the RNA from the gel to a solid support, such as nitrocellulose or a nylon membrane. The immobilized RNA is then probed with a labeled probe to detect RNA species complementary to the probe used. Northern blots are a standard tool of molecular biologists (J. Sambrook, J. et al., supra, pp 7.39-7.52 [1989]).

The term “Western blot” refers to the analysis of protein(s) (or polypeptides) immobilized onto a support such as nitrocellulose or a membrane. The proteins are run on acrylamide gels to separate the proteins, followed by transfer of the protein from the gel to a solid support, such as nitrocellulose or a nylon membrane. The immobilized proteins are then exposed to antibodies with reactivity against an antigen of interest. The binding of the antibodies may be detected by various methods, including the use of radiolabelled antibodies.

The term “antigenic determinant” as used herein refers to that portion of an antigen that makes contact with a particular antibody (i.e., an epitope). When a protein or fragment of a protein is used to immunize a host animal, numerous regions of the protein may induce the production of antibodies which bind specifically to a given region or three-dimensional structure on the protein; these regions or structures are referred to as antigenic determinants. An antigenic determinant may compete with the intact antigen (i.e., the “immunogen” used to elicit the immune response) for binding to an antibody.

The terms “specific binding” or specifically binding” when used in reference to the interaction of an antibody and a protein or peptide means that the interaction is dependent upon the presence of a particular structure (ie., the antigenic determinant or epitope) on the protein; in other words the antibody is recognizing and binding to a specific protein structure rather than to proteins in general. For example, if an antibody is specific for epitope “A,” the presence of a protein containing epitope A (or free, unlabelled A) in a reaction containing labelled “A” and the antibody will reduce the amount of labelled A bound to the antibody.

The term “transgene” as used herein refers to a foreign gene that is placed into an organism by, for example, introducing the foreign gene into newly fertilized eggs or early embryos. The term “foreign gene” refers to any nucleic acid (e.g., gene sequence) that is introduced into the genome of an animal by experimental manipulations and may include gene sequences found in that animal so long as the introduced gene does not reside in the same location as does the naturally-occurring gene.

As used herein, the term “vector” is used in reference to nucleic acid molecules that transfer DNA segment(s) from one cell to another. The term “vehicle” is sometimes used interchangeably with “vector.” Vectors are often derived from plasmids, bacteriophages, or plant or animal viruses.

The term “expression vector” as used herein refers to a recombinant DNA molecule containing a desired coding sequence and appropriate nucleic acid sequences necessary for the expression of the operably linked coding sequence in a particular host organism. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals.

Embryonal cells at various developmental stages can be used to introduce transgenes for the production of transgenic animals. Different methods are used depending on the stage of development of the embryonal cell. The zygote is the best target for micro-injection. In the mouse, the male pronucleus reaches the size of approximately 20 micrometers in diameter which allows reproducible injection of 1-2 picoliters (pl) of DNA solution. The use of zygotes as a target for gene transfer has a major advantage in that in most cases the injected DNA will be incorporated into the host genome before the first cleavage (Brinster et al., Proc. Natl. Acad. Sci. USA 82:4438-4442 [1985]). As a consequence, all cells of the transgenic non-human animal will carry the incorporated transgene. This will, in general, also be reflected in the efficient transmission of the transgene to offspring of the founder since 50% of the germ cells will harbor the transgene. Micro-injection of zygotes is the preferred method for incorporating transgenes in practicing the invention. U.S. Pat. No. 4,873,191 describes a method for the micro-injection of zygotes; the disclosure of this patent is incorporated herein in its entirety.

Retroviral infection can also be used to introduce transgenes into an animal. The developing embryo can be cultured in vitro to the blastocyst stage. During this time, the blastomeres can be targets for retroviral infection (Janenich, Proc. Natl. Acad. Sci. USA 73:1260-1264 [1976]). Efficient infection of the blastomeres is obtained by enzymatic treatment to remove the zona pellucida (Hogan et al., in Manipulating the Mouse Embryo, Cold Spring Harbor Laboratory Press, Cold Spring Harbor, N.Y. [1986]). The viral vector system used to introduce the transgene is typically a replication-defective retrovirus carrying the transgene (D. Jahner et al., Proc. Natl. Acad Sci. USA 82:6927-693 [1985]). Transfection is easily and efficiently obtained by culturing the blastomeres on a monolayer of virus-producing cells (Van der Putten, supra; Stewart, et al., EMBO J. 6:383-388 [1987]). Alternatively, infection can be performed at a later stage. Virus or virus-producing cells can be injected into the blastocoele (D. Jahner et al., Nature 298:623-628 [1982]). Most of the founders will be mosaic for the transgene since incorporation occurs only in a subset of cells that form the transgenic animal. Further, the founder may contain various retroviral insertions of the transgene at different positions in the genome that generally will segregate in the offspring. In addition, it is also possible to introduce transgenes into the germline, albeit with low efficiency, by intrauterine retroviral infection of the midgestation embryo (Jahner et al., supra [1982]). Additional means of using retroviruses or retroviral vectors to create transgenic animals known to the art involves the micro-injection of retroviral particles or mitomycin C-treated cells producing retrovirus into the perivitelline space of fertilized eggs or early embryos (PCT International Application WO 90/08832 [1990], and Haskell and Bowen, Mol. Reprod. Dev., 40:386 [1995]).

A third type of target cell for transgene introduction is the embryonal stem (ES) cell. ES cells are obtained by culturing pre-implantation embryos in vitro under appropriate conditions (Evans et al., Nature 292:154-156 [1981]; Bradley et al., Nature 309:255-258 [1984]; Gossler et al., Proc. Acad. Sci. USA 83:9065-9069 [1986]; and Robertson et al., Nature 322:445-448 [1986]). Transgenes can be efficiently introduced into the ES cells by DNA transfection by a variety of methods known to the art including calcium phosphate co-precipitation, protoplast or spheroplast fusion, lipofection and DEAE-dextran-mediated transfection. Transgenes may also be introduced into ES cells by retrovirus-mediated transduction or by micro-injection. Such transfected ES cells can thereafter colonize an embryo following their introduction into the blastocoel of a blastocyst-stage embryo and contribute to the germ line of the resulting chimeric animal (for review, See, Jaenisch, Science 240:1468-1474 [1988]). Prior to the introduction of transfected ES cells into the blastocoel, the transfected ES cells may be subjected to various selection protocols to enrich for ES cells that have integrated the transgene assuming that the transgene provides a means for such selection. Alternatively, the polymerase chain reaction may be used to screen for ES cells that have integrated the transgene. This technique obviates the need for growth of the transfected ES cells under appropriate selective conditions prior to transfer into the blastocoel.

The terms “overexpression” and “overexpressing” and grammatical equivalents, are used in reference to levels of mRNA to indicate a level of expression approximately 3-fold higher than that typically observed in a given tissue in a control or non-transgenic animal. Levels of mRNA are measured using any of a number of techniques known to those skilled in the art including, but not limited to Northern blot analysis. Appropriate controls are included on the Northern blot to control for differences in the amount of RNA loaded from each tissue analyzed (e.g., the amount of 28S rRNA, an abundant RNA transcript present at essentially the same amount in all tissues, present in each sample can be used as a means of normalizing or standardizing the mRNA-specific signal observed on Northern blots). The amount of mRNA present in the band corresponding in size to the correctly spliced transgene RNA is quantified; other minor species of RNA which hybridize to the transgene probe are not considered in the quantification of the expression of the transgenic mRNA.

The term “transfection” as used herein refers to the introduction of foreign DNA into eukaryotic cells. Transfection may be accomplished by a variety of means known to the art including calcium phosphate-DNA co-precipitation, DEAE-dextran-mediated transfection, polybrene-mediated transfection, electroporation, microinjection, liposome fusion, lipofection, protoplast fusion, retroviral infection, and biolistics.

The term “stable transfection” or “stably transfected” refers to the introduction and integration of foreign DNA into the genome of the transfected cell. The term “stable transfectant” refers to a cell which has stably integrated foreign DNA into the genomic DNA.

The term “transient transfection” or “transiently transfected” refers to the introduction of foreign DNA into a cell where the foreign DNA fails to integrate into the genome of the transfected cell. The foreign DNA persists in the nucleus of the transfected cell for several days. During this time the foreign DNA is subject to the regulatory controls that govern the expression of endogenous genes in the chromosomes. The term “transient transfectant” refers to cells which have taken up foreign DNA but have failed to integrate this DNA.

The term “calcium phosphate co-precipitation” refers to a technique for the introduction of nucleic acids into a cell. The uptake of nucleic acids by cells is enhanced when the nucleic acid is presented as a calcium phosphate-nucleic acid co-precipitate. The original technique of Graham and van der Eb (Graham and van der Eb, Virol., 52:456 [1973]), has been modified by several groups to optimize conditions for particular types of cells. The art is well aware of these numerous modifications.

As used herein, the term “selectable marker” refers to the use of a gene that encodes an enzymatic activity that confers the ability to grow in medium lacking what would otherwise be an essential nutrient (e.g. the HIS3 gene in yeast cells); in addition, a selectable marker may confer resistance to an antibiotic or drug upon the cell in which the selectable marker is expressed. Selectable markers may be “dominant”; a dominant selectable marker encodes an enzymatic activity that can be detected in any eukaryotic cell line. Examples of dominant selectable markers include the bacterial aminoglycoside 3′phosphotransferase gene (also referred to as the neo gene) that confers resistance to the drug G418 in mammalian cells, the bacterial hygromycin G phosphotransferase (hyg) gene that confers resistance to the antibiotic hygromycin and the bacterial xanthine-guanine phosphoribosyl transferase gene (also referred to as the gpt gene) that confers the ability to grow in the presence of mycophenolic acid. Other selectable markers are not dominant in that there use must be in conjunction with a cell line that lacks the relevant enzyme activity. Examples of non-dominant selectable markers include the thymidine kinase (tk) gene that is used in conjunction with tk⁻cell lines, the CAD gene which is used in conjunction with CAD-deficient cells and the mammalian hypoxanthine-guanine phosphoribosyl transferase (hprt) gene which is used in conjunction with hprt⁻cell lines. A review of the use of selectable markers in mammalian cell lines is provided in Sambrook, J. et al., Molecular Cloning: A Laboratory Manual, 2nd ed., Cold Spring Harbor Laboratory Press, New York (1989) pp.16.9-16.15.

As used herein, the term “cell culture” refers to any in vitro culture of cells. Included within this term are continuous cell lines (e.g., with an immortal phenotype), primary cell cultures, finite cell lines (e.g., non-transformed cells), and any other cell population maintained in vitro.

As used, the term “eukaryote” refers to organisms distinguishable from “prokaryotes.” It is intended that the term encompass all organisms with cells that exhibit the usual characteristics of eukaryotes, such as the presence of a true nucleus bounded by a nuclear membrane, within which lie the chromosomes, the presence of membrane-bound organelles, and other characteristics commonly observed in eukaryotic organisms. Thus, the term includes, but is not limited to such organisms as fungi, protozoa, and animals (e.g., humans).

As used herein, the term “in vitro” refers to an artificial environment and to processes or reactions that occur within an artificial environment. In vitro environments can consist of, but are not limited to, test tubes and cell culture. The term “in vivo” refers to the natural environment (e.g., an animal or a cell) and to processes or reaction that occur within a natural environment.

The term “test compound” refers to any chemical entity, pharmaceutical, drug, and the like that can be used to treat or prevent a disease, illness, sickness, or disorder of bodily function. Test compounds comprise both known and potential therapeutic compounds. A test compound can be determined to be therapeutic by screening using the screening methods of the present invention. A “known therapeutic compound” refers to a therapeutic compound that has been shown (e.g., through animal trials or prior experience with administration to humans) to be effective in such treatment or prevention.

The term “sample” as used herein is used in its broadest sense and includes environmental and biological samples. Environmental samples include material from the environment such as soil and water. Biological samples may be animal, including, human, fluid (e.g., blood, plasma and serum), solid (e.g., stool), tissue (e.g., cardiac tissue), liquid foods (e.g., milk), and solid foods (e.g., vegetables).

### DESCRIPTION OF THE INVENTION

The present invention relates to methods and compositions for tissue-restricted gene recombination. In particular, the present invention provides methods and compositions for tissue-restricted gene recombination in post-mitotic cells. The present invention further provides methods for gene recombination in post-mitotic cells comprising the delivery of a Cre recombinase to the target tissue to facilitate recombination in a desired target nucleic acid.

As described above, animal models of human disease can be generated by homologous recombination for germline loss-of-function mutations. However, embryonic-lethal phenotypes and systemic, indirect dysfunction can confound the use of knock-outs to elucidate adult pathophysiology. Site-specific recombination using Cre recombinase can circumvent these pitfalls, in principle, enabling temporal and spatial control of gene recombination. However, prior to the present invention, direct evidence was lacking for the feasibility of Cre-mediated recombination in post-mitotic cells and an efficient and effective method for such recombination was not available.

For example, a recent report using viral delivery of Cre in vivo to activate a loxP-tagged transgene did not demonstrate recombination in post-mitotic cells (Wang et al., Proc. Natl. Acad. Sci. 93, 3932 [1996]). In this study, recombination in neurons was unjustifiably extrapolated from PCR evidence for recombination after intracerebral injection, taken together with the presence of LacZ in neurons after parallel injections of virus encoding LacZ directly. The identity of cell types undergoing recombination was left unanswered for both the brain and the heart, which had low levels of recombination (i.e., the recombination may have occurred in glia or other stromal cells). Thus, Wang et al., do not prove or teach effective methods for recombination in post-mitotic cells and do not prove or teach efficient recombination in heart, even for mitotic cells. In contrast, the present invention demonstrates Cre-dependent activation of the CAG-CATZ transgene and knockout of specifically targeted exons, following adenoviral delivery of Cre recombinase to post-mitotic cells in vivo. These findings provide solutions to the problems of previous studies which failed to establish that post-mitotic cells were not refractory to recombination produced by Cre.

Further, the present invention provides transgenic technology plus viral gene transfer to achieve Cre-mediated recombination in a tissue-specific manner. For example, in one in vitro embodiment of the present invention, Cre driven by cardiac-specific α-myosin heavy chain (αMHC) sequences elicited recombination selectively at loxP sites in purified cardiac myocytes, but not cardiac fibroblasts. In vivo, this αMHC-Cre transgene elicited recombination in cardiac muscle, but not other organs, as ascertained by PCR analysis and localization of a recombination-dependent reporter protein. Adenoviral delivery of Cre in vivo provoked recombination in post-mitotic, adult ventricular myocytes. Recombination between loxP sites was not detected in the absence of Cre. These results demonstrate the feasibility of using Cre-mediated recombination to regulate gene expression in specific tissues (e.g., myocardium), with efficient induction of recombination even in terminally differentiated, post-mitotic cells. Moreover, delivery of Cre by viral infection provides a simple strategy to control the timing of recombination.

Both yeast (See e.g., O'Gorman et al., Science 251:1351 [1991]; Walters et al., Genes Dev. 10:185 [1996]; Dymecki, Proc. Natl. Acad. Sci. 93:6191 [1996]) and phage (See e.g., Sauer and Henderson, Proc. Natl. Acad. Sci. 85:5166 [1988]; Fukushige and Sauer, Proc. Natl. Acad. Sci. 89:7905 [1992]; Lakso et al., Proc. Natl. Acad. Sci. 89:6232 [1992]; Orban et al., Proc. Natl. Acad. Sci. 89:6861 [1992]; Baubonis and Sauer, Nucleic Acids Res. 21:2025 [1993]; Sauer, Methods Enzymol 225:890 [1993]; Van et al., Proc. Natl. Acad. Sci. 92:7376 [1995]; Smith et al., Nat. Genet. 9:376 [1995]; Metzger et al., Proc. Natl. Acad. Sci. 92:6991 [1995]; Gu et al., Science 265:103 [1994]; and Kühn et al., Science 269:1427 [1995]) site-specific recombinases have been used to achieve targeted recombination of mammalian genes. Thus, regulated expression of recombinase, in turn, can be used in principle to achieve tissue-restricted and/or stage-specific recombination to induce or inactivate predetermined genes.

Cre recombinase, from the P1 bacteriophage, has proven efficacious in mediating recombination in mammalian cells. The 38 kDa Cre protein functions as a site-specific recombinase, splicing DNA between specific 34 base-pair sequences known as loxP sites. Each loxP site contains a dyad of 13 base pairs, separated by an eight base pair spacer that gives 5′to 3′orientation to the motif. Cre is capable of catalyzing three forms of recombination: excision, inversion, and integration, dictated by the orientation of loxP sites relative to each other. Excision or inversion occur when the loxP sites exist on the same strand of DNA (as direct versus inverted repeats, respectively); insertion can occur, using loxP sites on separate strands.

Cre-mediated recombination can be regulated by controlling the timing or spatial distribution of Cre expression via tissue-specific promoters (Gu et al., supra), ligand-inducible promoters (Kühn et al., supra), and ligand-dependent Cre fusion proteins (Metzger et al., supra). For example, Kühn et al. deleted a DNA polymerase β gene, flanked by loxP sites (i.e., “floxed”), by expressing Cre in transgenic mice using the M×1 promoter, an interferon-dependent promoter. Densitometric analyses of Southern blots suggested that 100% of the pol β alleles in liver and spleen were deleted after induction of Cre expression. Additionally, 65% of the allele was lost in cardiac tissue as well. However, there was no indication that recombination occurred in post-mitotic cells in the heart tissue. The results relied on a Southern blot and do not establish whether recombination occurred in the post-mitotic myocytes, versus the abundant populations of interstitial fibroblasts (ie., mitotic cells) and other non-muscle cells that largely comprise the adult heart. For example, approximately 85% of the cells in the murine heart are not myocytes (Soonpaa et al., Am. J. Physiol. 40:H2183 [1996]). The same limitation was reported in a paper demonstrating the utility of Flp recombinase in transgenic mice, where a 30% recombination frequency in DNA extracted from skeletal muscle leaves substantial room for doubt concerning the identity of cell types involved (Dymecki, supra). Despite the attractiveness of site-directed recombination for applications affecting terminally differentiated lineages such as cardiac or skeletal muscle, prior to the present invention, there has not been a definitive method for site-directed recombination in an irreversibly post-mitotic muscle cell.

In certain embodiments, the present invention provides both a tissue-specific promoter of Cre recombinase and injection of recombinant adenoviruses (Wang et al., Somatic Cell Mol. Genet. 21:429 [1995]) that have been used to direct Cre expression in specific target tissue. Cre-mediated recombination was found to occur both in cultured cells and the intact adult tissues. These studies establish the utility of targeting Cre to specific tissues, to achieve gene recombination even in post-mitotic cells.

The capacity to trigger site-specific gene recombination provided by the present invention, with both temporal and spatial control, has definite advantages over methods in the art. Driven by tissue-specific control sequences, the Cre transgenes provided by the present invention catalyze site-specific gene rearrangement selectively in the desired target tissues. Moreover, the use of recombinant virus as an acute delivery vehicle for Cre allows for recombination triggered by Cre in post-mitotic cells. Because no prior work has provided a definitive method for Cre-mediated recombination that can be triggered in terminally differentiated, post-mitotic cells (e.g., muscle cells), the importance of the present investigation is clear. The adenoviral delivery methods provided by the present invention are especially well-suited for Cre-mediated recombination, given the capacity of recombinant adenoviruses for remarkably efficient gene transfer to many non-proliferating cells (See e.g., ventricular myocytes [Kirshenbaum et al., J. Clin. Invest. 92:381 (1993); and Kass-Eisler et al., Proc. Natl. Acad. Sci. 90:11498 (1993)]; and skeletal muscle [Stratford-Perricaudet et al., J. Clin. Invest. 90:626 (1992)]). More broadly, the present invention provides methods and compositions for drug testing and screening and the development of animal models for disease states (e.g., human disease states).

Thus, the present invention provides the ability to selectively eliminate certain genes from specific post-mitotic tissues, while preserving the remainder of the body in a wild-type state. Beyond merely surmounting the vexing problem of embryonic or early post-natal lethality, this specificity also provides a much-needed means to ensure that the consequences of a given gene deletion are due to the protein's function in the affected cells, and not an untoward, confounding systemic or indirect effect. Moreover, viral delivery of Cre provides a versatile means to generate an array of organ-specific knock-out mutations, as a rapid, less costly alternative to constructing and maintaining an array of tissue-specific Cre animals. The ability shown here, to evoke gene recombination selectively in specific tissues, even in terminally differentiated, post-mitotic tissues, dramatically expands the biological context in which gene targeting can be efficiently conducted.

In addition to the ability to generate models for disease states, the methods and compositions of the present invention find further use in drug testing and drug screening. For example, a gene in adult animals suspected of being involved in a disease state can be “knocked-out” by the methods of the present invention. Drugs (e.g., proteins, including growth factors, cytokines, hormones, and enzymes, or non-protein molecules) can be administered to the animal to determine if they are effective at compensating for the loss of gene function. In this manner, the Cre-dependent knockout animals will serve as a test or model system, in which the optimal type and dose of drug can be systematically characterized.

The present invention contemplates many other means of screening compounds. The examples provided herein are presented merely to illustrate examples of techniques available. One of ordinary skill in the art will appreciate that many other screening methods can be used.

### EXPERIMENTAL

The following examples are provided in order to demonstrate and further illustrate certain preferred embodiments and aspects of the present invention and are not to be read as limiting the scope thereof. One skilled in the art will appreciate the applicability of these methods and compositions to a wide variety of applications.

In the experimental disclosure which follows, the following abbreviations apply: ° C. (degrees Centigrade); rpm (revolutions per minute); IM (intramuscular); IP (intraperitoneal); IV (intravenous or intravascular); SC (subcutaneous); H₂O (water); aa (amino acid); bp (base pair); kb (kilobase pair); kD (kilodaltons); gm (grams); μg (micrograms); mg (milligrams); ng (nanograms); μl (microliters); ml (milliliters); mm (millimeters); nm (nanometers); μm (micrometer); M (molar); mM (millimolar); μM (micromolar); nM (nanomolar); U (units); V (volts); MW (molecular weight); μCi (microcurrie); sec (seconds); min(s) (minute/minutes); hr(s) (hour/hours); ab (antibody); IC₅₀ (50% inhibitory concentration); DTT (dithiothreitol); HCl (hydrochloric acid); MgCl₂ (magnesium chloride); KCl (potassium chloride); NaCl (sodium chloride); T3 (triiodothyronine); PAGE (polyacrylamide gel electrophoresis); SDS-PAGE (sodium dodecyl sulfate polyacrylamide gel electrophoresis); PBS (phosphate buffered saline [150 mM NaCl, 10 mM sodium phosphate buffer, pH 7.2]); PCR (polymerase chain reaction); PEG (polyethylene glycol); PMSF (phenylmethylsulfonyl fluoride); RT-PCR (reverse transcription PCR); SDS (sodium dodecyl sulfate); CMV (cytomegalovirus); PFU (plaque-forming units); IFN (interferon); MHC (myosin heavy chain); MOI (multiplicities of infection); X-gal (5-bromo-4-chloro-3-indolyl-β-D galactopyranoside); Tris (tris(hydroxymethyl)aminomethane); EDTA (Ethylenediaminetetraacetic Acid); w/v (weight to volume); v/v (volume to volume); Bio-Rad (BioRad, Richmond, Calif.); Beckman (Beckman Instruments, Fullerton, Calif.); Dupont-NEN (Dupont-NEN, Boston, Mass.); Wheaton (Wheaton Science Products); Boehringer Mannheim (Boehringer Mannheim, Indianapolis, Ind.); Amersham (Amersham, Arlington Heights, Ill.); Pierce (Pierce, Rockford, Ill.); Santa Cruz (Santa Cruz, Santa Cruz, Calif.); and Sigma (Sigma Chemical Co., St. Louis, Mo.).

Statistical analysis was performed using ANOVA and Scheffe's multiple comparison test, using a significance level of p<0.05.

### EXAMPLE 1

### Biochemical Assays

The present example describes some of the biochemical assays utilized during the development of the present invention.

Luciferase activity was monitored as the oxidation of luciferin in the presence of coenzyme A, using an Analytical Luminescence (Analytical Luminescence Laboratories, La Jolla, Calif.) model 2010 luminometer as described by Brand et al. (Brand et al., J. Biol. Chem. 268:11500 [1993]). LacZ activity was determined using chlorophenol red-β-D-galactosidase as substrate as described Eustice et al. (Eustice et al., Biotechniques 11:739 [1991]). Total protein was measured by the Bradford method (Bradford, Anal. Biochem. 72:248 [1976]). CAT activity was measured by the phase-extraction method (Seed and Sheen, Gene 67, 271 [1988]) with 25 mg of n-butyryl coenzyme A, 24 mM D-threo-di-chloroacetyl-1,2 [¹⁴C]chloramphenicol (DuPont-NEN), and 100 ml of lysate in 25 mM Tris-HCl, pH 7.4, using a Beckman LS6800 scintillation counter. For biochemical determinations of reporter gene activity in vivo, hearts were washed and homogenized for 15 sec using a Tissumizer (Wheaton), in 250 ml per 100 mg of 25 mM Tris phosphate, pH 7.8, 2 mM dithiothreitol, 2 mM EDTA, 10% glycerol, and 1% Triton X-100, containing 1 mg/ml Pefabloc SC (Boehringer Mannheim), 0.5 mg/ml EDTA, 10 mg/ml leupeptin, 10 mg/ml pepstatin, and 1 mg/ml aprotinin as protease inhibitors. Homogenates were centrifuged at 10,000×g for 10 min at room temperature, and the supernatants analyzed as described below for cultured cells.

### EXAMPLE 2

### Cardiac-Specific Cre Vector Mediates Selective Recombination

In these experiments, a cardiac-specific Cre expression vector was developed and used to mediate selective recombination in cultured ventricular muscle cells.

A. Construction of the αMHC-Cre Gene

To achieve cardiac myocyte-restricted expression of Cre recombinase, Cre cDNA was placed under the transcriptional control of the cardiac-specific αMHC promoter. The nuclear-localized Cre expression vector, pOG231 (provided by S. O'Gorman, Salk Institute for Biological Studies), was digested with SacI and HindIII, removing the CMV promoter but retaining the ATG start site and SV40 nuclear localization signal (i.e., the nuclear localization signal sequence: 5′-CCCAAGAAGAAGAGGAAGGTG-3′[SEQ ID NO: 1]; corresponding to amino acid sequence PKKKRKV [SEQ ID NO: 2]) in frame with the first codon of Cre cDNA (i.e., first codon sequence 5′-TTC-3′). The cardiac-specific murine α-myosin heavy chain (MHC) promoter (ac-5.5, provided by J. Robbins, University of Cincinnati) (Subramaniam et al., J. Biol. Chem. 266, 24613 [1991]) was ligated as a SacI-HindIII fragment 180 bp upstream from the nis-Cre translational start site. The resulting αMHC-Cre expression vector comprised the βMHC 3′untranslated region, αMHC promoter, αMHC non-coding exons 1 and 2 (with the corresponding introns), and an exon 3 splice acceptor sequence, located 180 bp upstream from the nls-Cre ATG start site.

The present invention contemplates the use of all cardiac-specific promoters. For example, promoters from other cardiac-restricted structural proteins such as the myosin light chain-2v (See e.g., Chen et al., J. Biol. Chem. 273:1252 [1998]) and myosin light chain-2a, or promoters from cardiac-restricted transcription factors such as Nkx-2.5 (See e.g., Sepulveda et al., Mol. Cell Biol. 18:3405 [1998]), and dHAND and eHAND (See e.g., Thomas et al., Dev. Biol. 196:228 [1998]), among others, will find use with the present invention. In various embodiments, the present invention contemplates the use of a range of tissue-specific promoters to allow expression in the desired tissue(s), at the desired level of expression, and in the species of interest. Such tissue-specific promoters are available in the scientific literature, with lists of regulatory regions found in various review articles or databases (See e.g., http://agave.humgen.upenn.edu/MTIR/TOC.html).

B. Cell Culture and Transfection

In parallel experiments, cultured ventricular myocytes and ventricular fibroblasts were co-transfected with the Cre-dependent reporter gene CAG-CATZ, described by Araki et al. (Araki et al., Proc. Natl. Acad. Sci. 92, 160 [1995]), together with the CMV promoter alone, CMV-driven Cre, αMHC promoter alone, or αMHC-driven Cre promoter. The results obtained for the cultured ventricular myocytes and ventricular fibroblasts were then compared.

Primary cultures of 2 day post-natal Sprague-Dawley rat cardiac myocytes and cardiac fibroblasts were prepared using enzymatic dissociation, Percoll (Pharmacia Fine Chemicals, Piscataway, N.J.) purification, and serum-free medium containing Dulbecco's modified Eagle's medium:Ham's medium F12 (1:1), 5 mg/ml transferrin, 1 nM Na₂SeO₄, 1 nM LiCl, and 25 mg/ml ascorbic acid (See e.g., Abdellatif et al., J. Biol. Chem. 269, 15423 [1994]). Cells were plated at a density of 2×10⁵ cells per 7 mm well for adenoviral infection and 9×10⁵ cells per 35 mm well for DNA-mediated gene transfer. Transfection of neonatal cardiac myocytes and cardiac fibroblasts was carried out using lipofectamine, 1.0 mg of the loxP-tagged CAG-CATZ reporter (Araki et al., Proc. Natl. Acad. Sci. 92, 160 [1995]), 0.5 mg of an SV40-driven luciferase reporter gene to correct for transfection efficiency, and 2.0 mg of the test vector (pcDNA3, the CMV promoter alone; OG231, CMV promoter driving Cre recombinase; p1120, the αMHC promoter alone; or αMHC promoter driving Cre). Cells were incubated for 4.5 hr at 37°C. in the lipofectamine:DNA solution, for 8 hr in Dulbecco's modified Eagle's medium:Ham's (DMEM:Ham's) medium F12 (1:1) supplemented with 5% horse serum, and for 60 hr in serum free medium, with the addition of 1 nM T3.

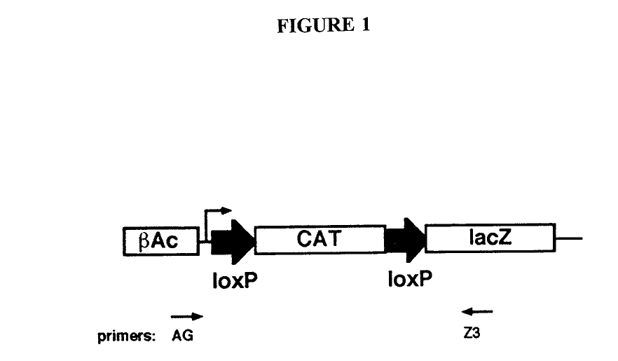

CAG-CATZ harbors a chloramphenicol acetyltransferase (CAT) gene flanked by loxP sites and driven by the chicken β-actin promoter (Araki et al., supra). As shown in FIG. 1, the E. coli β-galactosidase gene (lacZ) is located downstream of CAT. FIG. 1 provides a schematic of pCAG-CATZ, with two PCR primers, indicated as “AG” and “Z3,” that flank the paired loxP sites and internal CAT gene. In the absence of Cre, the CAT gene prevents read-through expression of lacZ. Conversely, when the CAT gene is excised by Cre-mediated recombination between the tandem loxP sites, lacZ becomes positioned adjacent to the β-actin promoter, permitting lacZ expression.

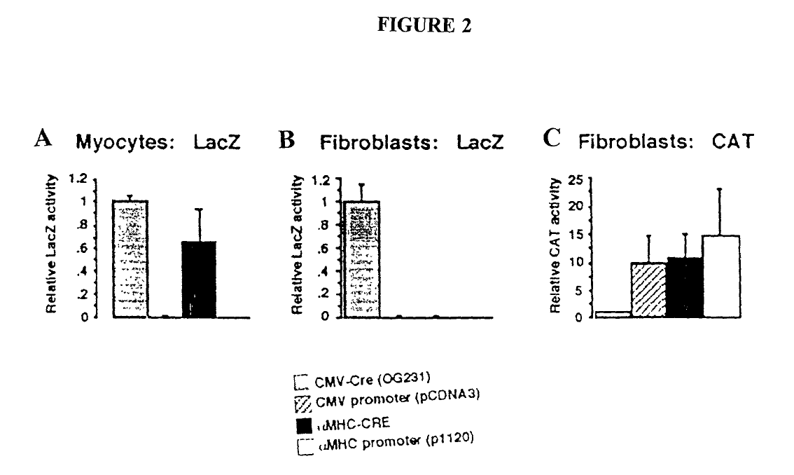

Results of the reporter gene expression for the various constructs are shown in FIGS. 2A, B, and C. In FIG. 2, the results are shown for CMV-Cre (grey), CMV promoter (hatched), αMHC-Cre (black), and αMHC promoter (white). FIG. 2A shows neonatal rat ventricular myocytes ventricular fibroblasts that were co-transfected with 1 mg pCAG-CATZ and 0.5 mg of an SV40-driven luciferase reporter gene (to correct for transfection efficiency), in the presence or absence of a Cre expression vector, as shown to the right of FIG. 2C. Cells were harvested 60 hr after transfection. The results shown in this figure (mean±standard error, n=13 for each condition shown), were adjusted for the co-transfected luciferase gene, and are expressed relative to CMV-Cre-transfected cells. Induction of lacZ by αMHC-Cre in cardiac muscle cells was roughly two-thirds of that obtained with the constitutive CMV-Cre vector. FIG. 2B shows the results for cardiac fibroblasts that were co-transfected as described for FIG. 2A. In contrast to the myocytes, lacZ expression was induced only by the CMV-Cre construct, and not the αMHC-driven Cre gene for these cells. FIG. 2C shows the results obtained with transfected cardiac fibroblasts that also were analyzed for CAT expression. As indicated in the Figure, the loxP-flanked CAT gene was down-regulated 90% by CMV-Cre, relative to cells co-transfected with pCAG-CATZ plus the CMV promoter.

As shown in the panels of FIG. 2, the CMV promoter was able to direct Cre-mediated recombination effectively in both cell types. In contrast, αMHC-Cre was selectively active, provoking gene recombination only in the ventricular myocytes. In addition, adequate transfection of fibroblasts was verified both by the co-transfected luciferase control, and by the capacity of CAG-CATZ to function in this background, as verified by CAT activity of the unmodified CAG-CATZ reporter in FIG. 2C. These results clearly show that Cre-mediated recombination can occur in neonatal ventricular myocytes using the methods and compositions of the present invention.

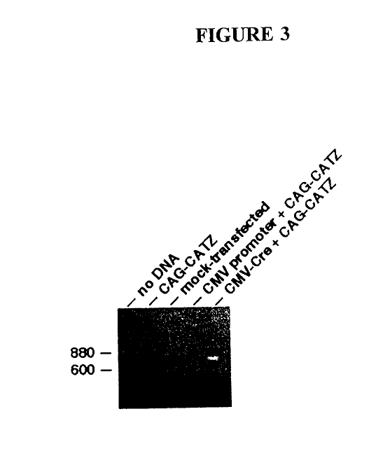

To validate the above results, Cre-mediated recombination at the loxP sites was assessed using the polymerase chain reaction (PCR). To demonstrate Cre-mediated recombination after transient transfection, myocytes were harvested from culture dishes using 0.02% trypsin in phosphate-buffered saline, and were incubated in 100 mM Tris-HCl, pH 7.5, 10 mM EDTA, 0.5% sodium dodecyl sulfate, 1 mg/ml proteinase K for 1 hr at 56°C. DNA was extracted in phenol/chloroform and was ethanol-precipitated. Oligonucleotides were synthesized corresponding to the 3′end of the chicken β-actin promoter (i.e., the “AG2” primer; 5′-CTGCTAACCATGTTCATGCC-3′[SEQ ID NO: 9]) and 5′end of the lacZ gene (i.e., the “Z3” primer; 5′GGCCTCTTCGCTATTACG-3′ [SEQ ID NO: 10]). PCR analysis was performed using Taq polymerase and 1 mg of DNA from each myocyte transfection in a 50 ml reaction mixture containing 500 mM KCl, 100 mM Tris-HCl, pH 9.0, 1.5 mM MgCl₂, 1% Triton X-100, and 0.6 mM dNTPs. DNA was denatured at 94°C. for 1 min; primers were annealed at 60°C. for 1 min and extended at 72°C. for 1 min for 32 cycles. The elongation time for the PCR runs was selected to prevent amplification of the intact, 2000 base-pair segment, while permitting amplification of the recombined, 690 base-pair segment (See e.g., FIG. 3).

As shown in FIG. 3, no recombination was detected when cardiac myocytes were co-transfected with CAG-CATZ and the control expression vector containing the CMV promoter alone. Thus, the appearance of LacZ protein and the predicted recombination event were each contingent on the delivery of Cre recombinase. Spontaneous recombination, while theoretically possible between the paired loxP sites, occurs under these conditions, if at all, at a frequency too low to be detected by PCR. The results in FIG. 3 establish the Cre-dependence and appropriate size for the recombined gene for in vivo work, described below.

The examples provided above utilize loxP recombination target sequences. However, the present invention is not limited to the use of these particular target sequences. A variety of additional recombination target sites are known in the art including, but not limited to, loxP2, loxP3, loxP23, loxP511, loxB, loxC2, loxL, loxR, loxA86, loxΔ117, frt, dif, flp and att, which will find use with the present invention.

### EXAMPLE 3

### The αMHC-Cre Transgene Directs Cardiac-Restricted Recombination in CAG-CATZ Mice

To establish the feasibility of cardiac-restricted recombination in vivo, the αMHC-Cre construct was used to generate transgenic mice. In addition, Cre-dependent reporter mice were generated, utilizing the chimeric CAG-CATZ gene described above. The αMHC-Cre gene was excised from the plasmid vector backbone using SacI and KpnI. A 6.9 kbp fragment of pCAG-CATZ (Araki et al., supra), containing the β-actin promoter, loxP-tagged CAT gene, and adjacent lacZ gene, was excised using SalI and PstI. DNA fragments were separated by electrophoresis through 1% agarose, and purified using Qiaex II reagents (Qiagen, Chatsworth Calif.). The linear αMHC-Cre and CAG-CATZ genes were microinjected separately into the pronuclei of FVB/N zygotes using standard methods (See e.g., Taketo et al., Proc. Natl. Acad. Sci. 88, 2065 [1991]), at a concentration of 2 ng/ml in 10 mM Tris HCl (pH 7.4), 0.1 M EDTA, and injected embryos were transferred to pseudopregnant ICR females.

Transgene-positive mice were identified by PCR, using primers specific for each construct, respectively. The presence of the CAG-CATZ transgene was assessed by amplification of genomic DNA from tail samples, using a sense primer at the 5′ end and antisense primer for the mid-portion of CAT cDNA, respectively (5′-CAGTCAGTTGCTCAATGTACC-3′[SEQ ID NO: 3]; and 5′-ACTGGTGAAACTCACCGA-3′[SEQ ID NO: 4]), and resulting in a 320 bp band. The presence of the transgenes was also investigated from DNA samples isolated from heart, liver, lung, skeletal muscle (quadriceps), or spleen at the time of necropsy. Primers were annealed at 60°C. for 1 min and extended at 72°C. for 1 min for 30 cycles. To identify αMHC-Cre transgenic mice, a sense primer positioned at the 3′ end of intron 2 of the αMHC promoter (5′-ATGACAGACAGATCCCTCCTATCTCC-3′ [SEQ ID NO: 5]) was used with an antisense primer at the 5′end of the Cre coding region (5′-CTCATCACTCGTTGCATCATCGAC-3′[SEQ ID NO: 6]), to amplify a 300 bp fragment. The PCR runs were conducted as described above, with the exceptions being that the primers were annealed at 59°C. for 1 min, with extension at 72°C. for 1 min, for 30 cycles of amplification. As a positive control, β-casein was amplified, using the following primers (sense, 5′-GATGTGCTCCAGGCTAAAGTT-3′ [SEQ ID NO: 7]; and antisense, 5′-AGAAACGGAATGTTGTGGAGT-3′[SEQ ID NO: 8]).

Two of 21 potential Fo Cre-dependent reporter mice carried the CAG-CATZ gene, while nine of 38 potential F₀ αMHC-Cre construct mice bore the αMHC-Cre gene. Of these mice, the CAG-CATZ founders designated as 2104 and 2112 and the αMHC-Cre founder designated as 2182 were arbitrarily selected for further characterization. To establish that the CAG-CATZ reporter was transcriptionally competent in adult myocardium, tissue extracts from F₁ progeny of CAG-CATZ parents were analyzed for expression of the CAT gene. As expected, the loxP-flanked CAT gene was highly active in myocardium and other organs of CAG-CATZ⁺mice, whereas the lacZ gene, distal to the second loxP site, was silent.

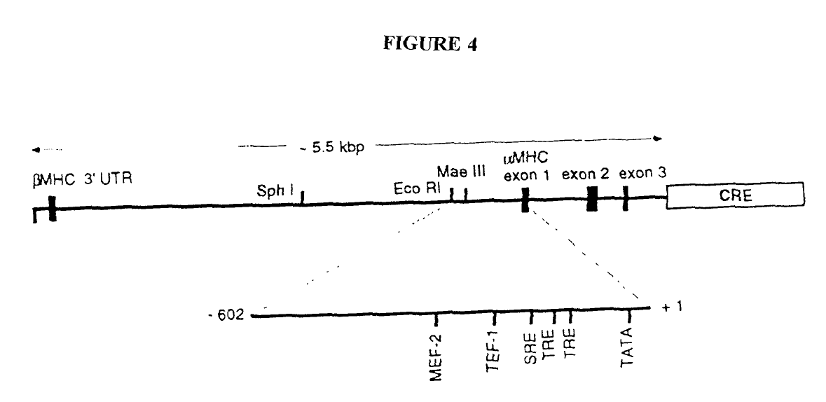

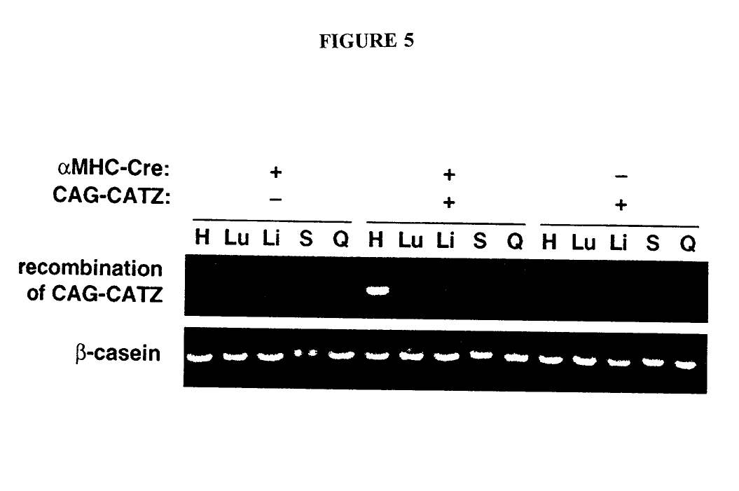

To test for Cre-mediated recombination of the CAG-CATZ target gene, CAG-CATZ F₁ mice and αMHC-Cre F₀ mice were mated. As shown by PCR analysis in FIG. 5, the αMHC-Cre and CAG-CATZ transgenes each were inherited by roughly half the progeny, and 2 of the 8 litter-mates received both genes. FIG. 4 shows the structure of the αMHC-Cre construct, while FIG. 5 presents the PCR analysis of representative litter-mates from the above mating. To define the genotype of each animal, tail DNA was analyzed for the presence of the αMHC-Cre and CAG-CATZ constructs, using PCR primers and conditions described above. Tissue DNA then was analyzed, as described in Example 2, for amplification of the recombination-dependent 690 bp fragment of CAG-CATZ, versus β-casein as a positive control. Myocardial recombination of the CAG-CATZ gene, assayed using the primers shown in FIG. 1, occurred only in the αMHC-Cre⁺/CAG-CATZ⁺double transgenic mice as shown in FIG. 5. Recombination was found to be cardiac-restricted and contingent on the presence of both transgenes concomitantly. No background or spontaneous recombination was detected.

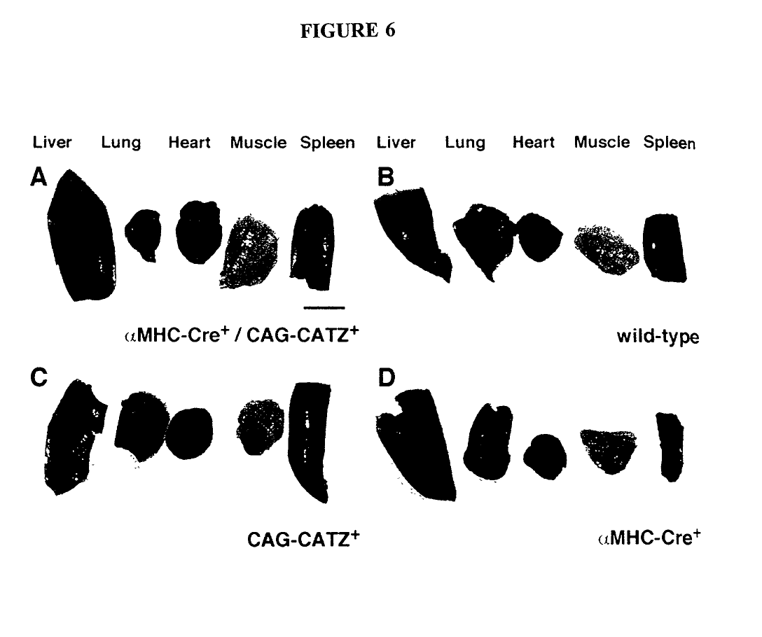

To demonstrate the functional consequences of Cre-dependent recombination, all 8 litter-mates were also analyzed by whole-organ X-gal staining of heart, liver, lung, skeletal muscle (quadriceps), and spleen as described below. At 6 weeks of age, the animals were sacrificed and heart, liver, lung, skeletal muscle (quadriceps), and spleen tissues were harvested. The tissues were fixed and incubated for 3 hr with 1 mg/ml X-gal. The stained tissues are shown in FIGS. 6A-D, with the horizontal bar representing 5 mm. The induction of LacZ activity was specific to myocardium (blue) and was contingent on the presence of the αMHC-Cre⁺/CAG-CATZ⁺genotype as shown in FIG. 6A as compared to 6B-D. Because all of the tissues tested in CAG-CATZ-mice contained substantial CAT activity, the expression of lacZ selectively in myocardium cannot be ascribed to tissue-restricted transcription of the Cre-dependent reporter gene. Conversely, CAT activity in myocardium was reduced ˜90% in αMHC-Cre⁺/CAG-CATZ⁺mice, relative to expression in mice harboring CAG-CATZ alone. A localized region of X-Gal staining extrinsic to the lung in FIG. 6A is consistent with the previously noted activity of the αMHC promoter in pulmonary veins (Subramaniam et al., supra).

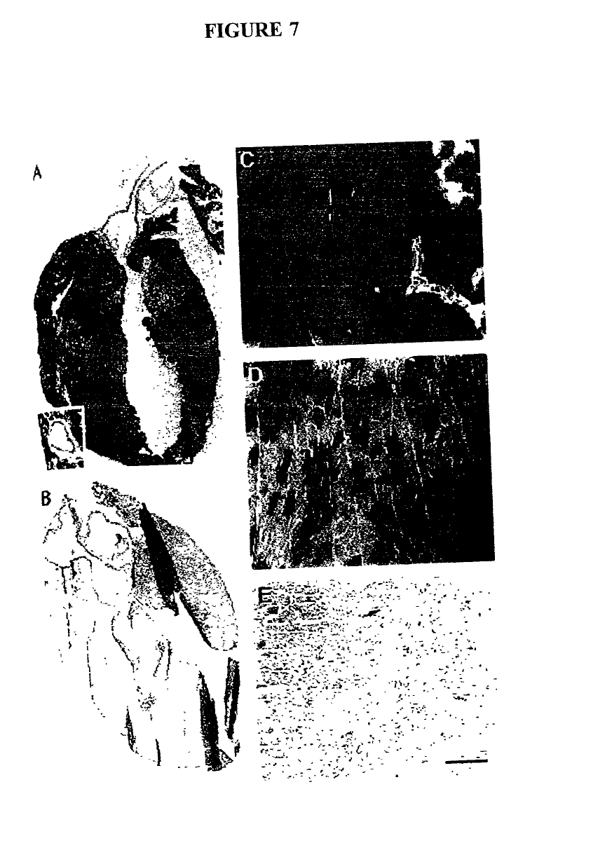

To substantiate that Cre-dependent recombination had in fact occurred in ventricular myocytes themselves, induction of LacZ protein in myocytes was corroborated by both histochemical staining as shown in FIGS. 7A-E and described in Example 8, and by immunolocalization. As shown in FIG. 7, lacZ induction was contingent on co-inheritance of the αMHC-Cre and CAG-CATZ genes and, by comparison to the prevalence in ventricular myocytes, was not detected in cardiac valves, coronary vessels, or the aorta (αMHC-Cre⁺/CAG-CATZ⁺[FIGS. 7A, C, and D]; αMHC-Cre⁺/CAG-CATZ⁺[FIGS. 7B and E]). Mice were derived from αMHC-Cre founder animal 2176, except for the mouse illustrated in FIG. 7D, which was derived from a lower-prevalence founder mouse designated 2177. The bar in the lower right corner represents 250 mm (A and B); 125 mm (A insert); and 50 mm (C, D, E). Roughly 90% of ventricular myocytes were LacZ-positive, for the sample illustrated in FIG. 7C. Other αMHC-Cre strains resulted in differing degrees of lacZ activation (e.g., FIG. 7D), which were unrelated to copy number. While the present invention is not limited to any particular mechanism of action, and an understanding of the mechanism is not required to practice the present invention, it is suggested that these results are consistent with insertional and positional effects or, potentially, differential methylation.

### EXAMPLE 4

### Viral Gene Transfer In Vivo

Because the αMHC promoter becomes transcriptionally active shortly after birth, in part resulting from the post-natal rise in thyroid hormone expression (Subramaniam et al., supra; and Subramaniam et al., J. Biol. Chem. 268, 4331 [1993]), additional experimentation was conducted to supplement the foregoing experiments (i.e., the experiments in Examples 1-3) to demonstrate the feasibility of Cre-mediated recombination in post-mitotic ventricular muscle.

The present Example provides several examples of viral systems for delivery of Cre in vivo. However, the present invention is not limited to these particular illustrative examples, and contemplates that any delivery system that provides efficient transfer to the desired tissue will find use with the present invention. For example, it is contemplated that the HVJ (Sendai virus) liposome method (See e.g., Isaka et al., Exp. Nephrol. 6:144 [1998]; and Yonemitsu et al., Int. J. Oncol. 12:1277 [1998]) and the use of lentiviruses (See e.g., Miyoshi et al., Proc. Natl. Acad. Sci. 94:10319 [1997]) or foamy virus (Russell and Miller, J. Virol. 70:217 [1996]) will dins use with the present invention. It is noted that, to date, prior to the development of the present invention, non-viral methods of delivery to certain tissues (e.g., cardiac muscle) have worked poorly in vivo, despite high efficiencies in vitro.

A) Adenoviral Delivery

In preparation for adenoviral delivery of Cre/lox reagents to adult myocardium in vivo, pilot studies first were performed with a recombinant virus encoding a nuclear-localized LacZ protein under the control of the CMV promoter (Ad5/CMV/nls-lacZ). Construction of Ad5/CMV/nls-lacZ, which directs the expression of E. coli lacZ, fused to the nuclear localization signal of SV40 large T antigen, was as described by French et al. (French et al., Circulation 90, 2402 [1994]). Ad5/CMV was created by subcloning the 1276 bp BglII-PvuII fragment containing the CMV immediate-early promoter, polylinker, and bovine growth hormone polyadenylation site from pcDNA3 (Invitrogen Corp., San Diego, Calif.) between the unique Bg/II and Klenow-blunted ClaI sites of the adenoviral shuttle vector pDE1sp1B as described by Bett et al. (Bett et al., Proc. Natl. Acad. Sci. 91, 8802 [1994]).

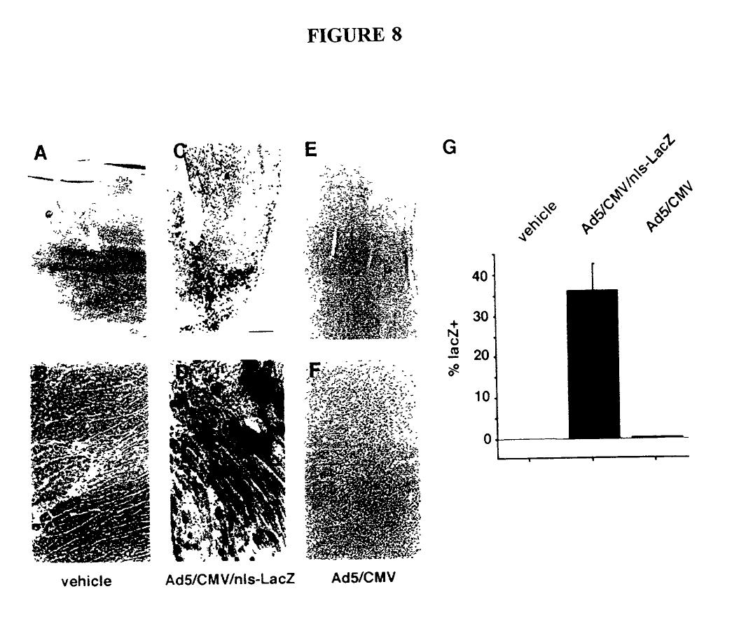

Given the small size of the adult mouse heart, a heart rate of 450-600 contractions per minute, and the objective of maximizing the reproducibility for gene delivery, injections were performed under direct visualization, following a median sternotomy, exteriorization of the heart, and transient immobilization. In this experiment, five mice were injected with Ad5/CMV/nls-lacZ, two were injected with the empty vector carrying only the CMV promoter (Ad5/CMV), and two were injected with diluent. All 9 of the mice were sacrificed 7 days post injection. To characterize the extent and reproducibility of gene delivery to adult mouse myocardium, four to five sections, approximately 250 μm apart, per heart were analyzed. The data obtained are shown in FIGS. 8A-G, where 8A and 8B show samples treated with vehicle injection; 8C and 8D with Ad5/CMV/nls-lacZ; and 8E and 8F with Ad5/CMV. FIG. 8G shows the proportion of LacZ-positive cells relative to total cell number in an immediately adjacent hematoxylin-eosin-stained section, scoring a 900×1300 mm area at the site of injection (range, 1017-1587 cells). For this experiment, lacZ expression was not contingent on Cre-mediated recombination. In FIG. 8, the bar represents 500 mm for panels A, C, and E; and 100 mm for panels B, D, and F. As shown in FIG. 8, all hearts injected with Ad5/CMV/nls-lacZ were positive for lacZ, by X-gal staining, in 3 or more of these widely spaced sections (i.e., FIGS. 8C and D). However, no LacZ activity was detected, after injection of the diluent (ie., FIGS. 8A and B) or Ad5/CMV (FIGS. 8E and F). The percentage of LacZ-positive nuclei averaged 36±11% (n=5) in the region of injection and ranged between 22% to 55% (FIG. 8G). In these samples, a mononuclear infiltrate was present in the area of injection for hearts injected either with Ad5/CMV/nls-lacZ or Ad5/CMV, consistent with evidence that viral antigens (in addition to virally delivered protein) evoke an immune response (Yang et al., Gene Ther. 3, 137 [1996]).

B. Adeno-associated Vector Delivery

The present invention further contemplates the use of adeno-associated virus vectors for the transfer of Cre to tissues (e.g., post-mitotic cells). Adeno-associated viruses (AAVs) are non-pathogenic viruses that are widespread in the human population. AAV vectors have been shown to transduce brain, skeletal muscle, and liver efficiently (See e.g., Kay et al., Proc. Natl. Acad. Sci. 94:12744 [1997]; and Fisher, Nature Med. 3:306 [1997]). Methods for transferring genes using AAVs to a variety of tissues are taught by International Patent Application WO 97/12050 (heart and vasculature); Kourtis et al., Modern Pathology 8:33A [1995] (cardiac tissue); March et al., European Heart Journal 13:218 [1992] (vascular smooth muscle); and Nahreini et al., Gene 119:265 [1992]; each of which is incorporated by reference herein in its entirety.

### EXAMPLE 5

### Adenoviral Delivery of Cre Triggers Recombination in Cultured Cardiac Myocytes

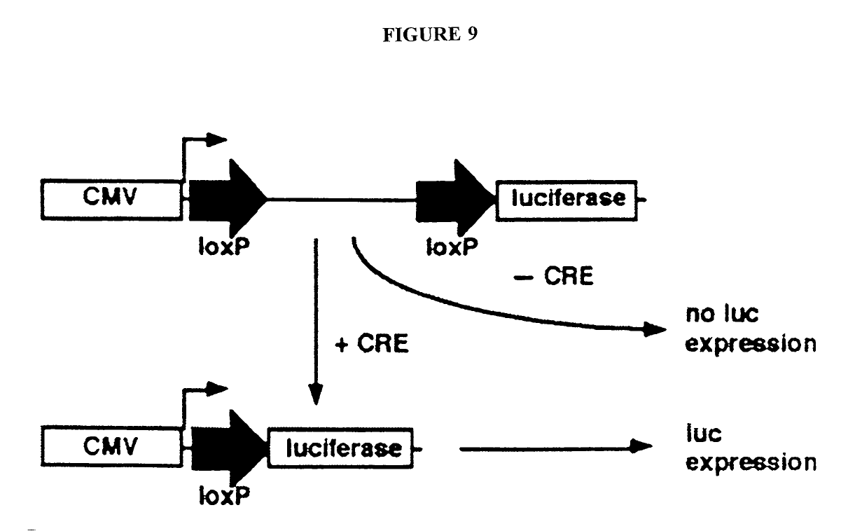

This Example demonstrates that adenoviral delivery of Cre to cultured cardiac myocytes triggers recombination. In these experiments, neonatal rat ventricular myocytes were co-infected with viruses bearing the CMV-driven Cre recombinase (AdCre) and a CMV-driven, recombination-dependent luciferase gene (AdMA19) (Anton and Graham, supra), as shown in FIG. 9. In this figure, the spacer interposed between the loxP sites precludes efficient luciferase expression in the absence of the Cre recombinase.

The resulting plasmid (pDE1sp1B/CMV) was then cotransfected with pJM17 into permissive 293 host cells to generate Ad5/CMV by standard methods (See e.g., Graham and Prevec, “Manipulation of adenovirus vectors” In Gene Transfer and Expression Protocols, Vol. 7, E. J. Murray ed., Humana Press, Inc., Totowa, N.J., pp 109-127 [1991]). AdCre (containing the CMV-driven Cre recombinase) and AdMA19 (containing the firefly luciferase reporter gene separated from the CMV promoter by a loxP-flanked 1.3 kilobase-pair spacer) (described by Anton and Graham; Anton and Graham, J. Viro. 69, 4600 [1995]) were generously provided by Dr. Frank Graham, McMaster University; the spacer region of AdMA19 contains stop codons in all three reading frames. All adenoviruses were propagated in 293 cells, harvested, cesium chloride-purified, and titered as described by Graham and Prevec (Graham and Prevec, supra). Cardiac cells were infected with recombinant adenoviruses, after 24 hours in serum-free medium, for 4 hr at multiplicities of infection (MOI) ranging from 0 to 300 PFU (plaque forming units) per cell, using 200 μl of virus in Dulbecco's modified Eagle's medium per well. The viral solution then was aspirated from each culture and replaced with 1 ml of serum free media for an additional 48 hr incubation period.

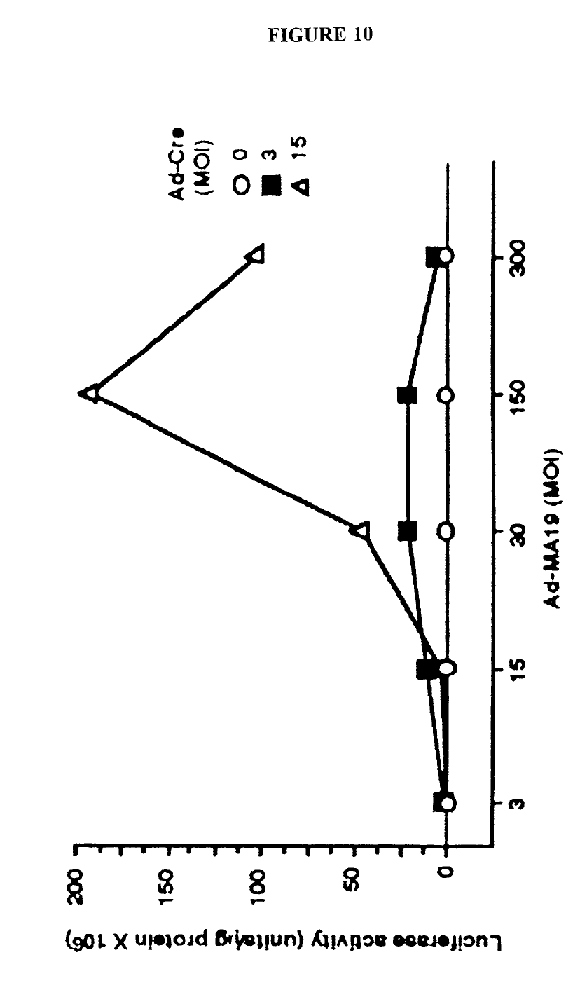

The multiplicities of infection ranged from 3 to 300 for each virus. As shown in FIG. 10, luciferase activity was contingent on the presence of both AdCre and AdMA19, verifying effective suppression of the reporter by the loxP-flanked spacer region inserted between the CMV promoter and luciferase gene. In this Figure, each determination shown was the mean of three separate cultures. Luciferase activity was maximal at 15 PFU/cell of AdCre and 150 PFU/cell of the reporter gene (193±41×10⁶ light units/μg protein, 225-fold greater than the luciferase produced in the absence of Cre at this concentration of AdMA19). Low-level expression of luciferase by this vector in the absence of Cre is ascribed to spontaneous recombination between the two homologous loxP sites (Anton and Graham, supra). While the present invention is not limited by any particular mechanism, and an understanding of the mechanism is not necessary to practice the present invention, it is suggested that luciferase activity was decreased at higher total concentrations of virus than those illustrated, due to cytopathic affects and/or promoter competition.

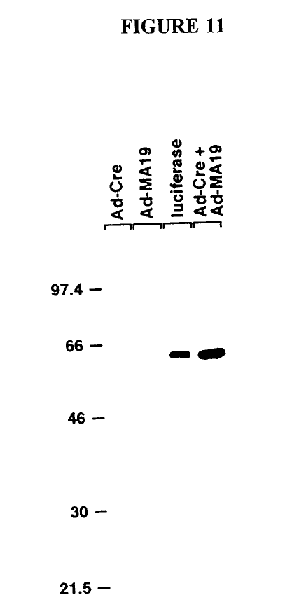

Lysates of myocytes infected with AdCre, AdMA19, or both in combination, were analyzed using Western blot analysis. For immunodetection of luciferase protein, cardiac myocytes were infected as described above with AdCre, AdMA19, or AdMA19; plus AdCre at a MOI of 15 for each virus. Cell lysates (50 μg of protein per lane) were resolved by electrophoresis in a 10% SDS-PAGE gel, in parallel with 300 ng of purified firefly luciferase (Boehringer Mannheim) as a positive control. Proteins were transferred electrophoretically to a 2 μm nylon membrane (Bio-Rad), using 0.1 M cyclohexylaminopropane sulfonic acid:10% methanol. The membrane was rinsed with buffer D (500 mM NaCl, 20 mM Tris-HCl, pH 7.5, 0.3% Tween 20) and incubated for 1 hour at room temperature with a polyclonal rabbit antibody directed against luciferase (Cortex, San Leano, Calif.), using a 1:4000 dilution in buffer D plus 0.2% fatty acid-free bovine serum albumin. The membrane then was washed for 30 min at room temperature with 3×50 ml of buffer D containing 3% Tween-20, incubated for 1 hr at room temperature with secondary antibody (horseradish peroxidase-conjugated donkey antibody against rabbit IgG; Amersham NA 934), using a 1:10,000 dilution in buffer D plus 0.2% fatty acid-free bovine serum albumin, and washed with 3×50 ml of buffer D plus 3% Tween-20. Bound antibody was detected by chemiluminescence, using ECL reagents (Amersham). The results are shown in FIG. 11. As shown in FIG. 11, luciferase protein was detected as a 62 kDa band, identical to the authentic protein standard, exclusively in cultures infected with AdCre and AdMA19 in tandem.

### EXAMPLE 6

### In Vivo Delivery of Cre Triggers Recombination in Post-Mitotic Ventricular Muscle

This Example demonstrates the effectiveness of the present invention for triggering recombination, in vivo, in post-mitotic muscle. For in vivo gene transfer to mouse myocardium, mice were anesthetized by intramuscular injection using 10 μl/gm of 4 mg/ml pentobarbital sodium in 20% ethanol, supplemented with 3 μl/gm as needed. The mice were intubated using a PE-90 endotracheal tube and ventilated using a small animal Harvard respirator (rate, 90-100 per min; tidal volume, 3-5 ml). Connection of the respirator to the PE-90 tubing was formed with PE-160 tubing to ensure a loose fit and prevent barotrauma by lung overexpansion as known in the art (See e.g., Michael et al., Am. J. Physiol. 269, H2147 [1995]). The skin overlying the sternum was incised, midline vessels of the sternum cauterized using bovie forceps, and the sternum divided. The sternum was retracted using 6-0 ligatures, and the heart was gently exteriorized using Crile forceps at the apex. Care was taken not to damage the lungs or great vessels in the process. The heart was briefly immobilized between the thumb and index finger of the operator and was injected at the apex over ˜2 seconds with 100 μl of phosphate-buffered saline (130 mM NaCl, 15 mM Na₂HPO₄, 15 mM NaH₂PO₄, 1 mM MgCl₂, and 1 mM CaCl₂) containing the virus, using a Hamilton syringe equipped with a 31-gauge needle. The heart was then returned to the thoracic cavity and the sternum closed. Over the next 15-30 min, upon return of spontaneous movement, the mice were removed from the ventilator and provided with 100% O₂, with spontaneous respiration resuming within 15-30 seconds. Body temperature was maintained during these procedures using a heat lamp, and 100% O₂ was provided through a nose cone.

For initial validation studies, 12 week-old ICR mice (35-40 gm, Harlan Sprague Dawley, Inc., Houston, Tex.) were inoculated with 10¹⁰ PFU of Ad5/CMV/nls-lacZ (n=5) versus Ad/CMV (n=2) or the diluent (n=2), and were sacrificed at 7 days. For the AdCre plus AdMA19 co-injection studies, ICR mice were injected with 6×10⁷ PFU of AdCre (n=2), 6×10⁷ PFU of AdMA19 (n=3), both viruses each at 6×10⁷ PFU (n=7), or 6×10⁷ PFU of Ad/CMV/nls-lacZ (n=2). For Cre activation of CAG-CATZ in transgenic mice, 9.5 week-old mice were injected with 2×10⁸ PFU of AdCre (n=3) or of Ad/CMV (n=2).

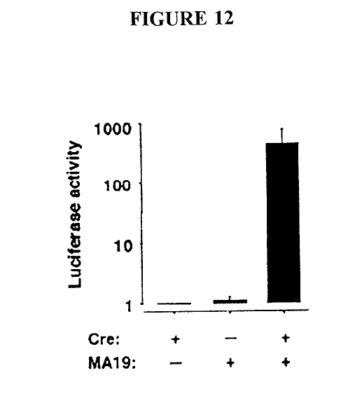

To test for Cre-mediated recombination after adenoviral injection of adult mouse myocardium, mice were injected with AdCre alone (n=2), AdMA19 (n=3), or both in concert (n=5). Mice were sacrificed 7 days after injection, and luciferase activity was assayed, as described in Example 1. The data are shown in FIG. 12. As shown in FIG. 12, luciferase production was detected in all 5 hearts that received the Cre virus and Cre-dependent luciferase gene (p=0.0001 versus only AdCre; p=0.0004 versus only AdMA19); no significant production above the machine reagent blank was seen in any heart that received either virus alone.

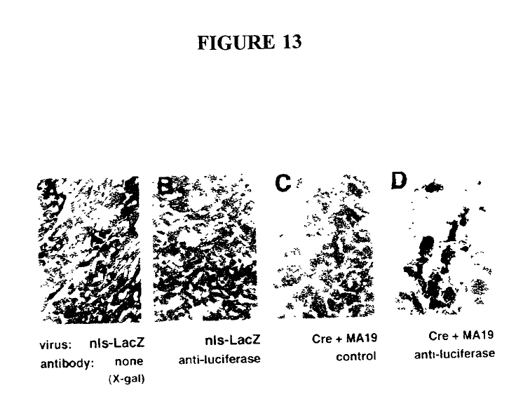

To verify the site of Cre-mediated recombination, immunohistochemistry for luciferase protein in the injected hearts also was performed as shown in FIGS. 13A-D. Two mice co-injected with AdCre plus AdMA19, and two with Ad5/CMV/nls-lacZ, were sacrificed at 3 days to minimize the complication associated with the presence of infiltrating cells in the area of injection. Hearts were sectioned as previously described and were stained for luciferase with the antibody used for the Western blot described in Example 5. Localization of luciferase signal to terminally differentiated adult myocytes, among other cells, was confirmed in the area of injection for multiple adjacent sections.

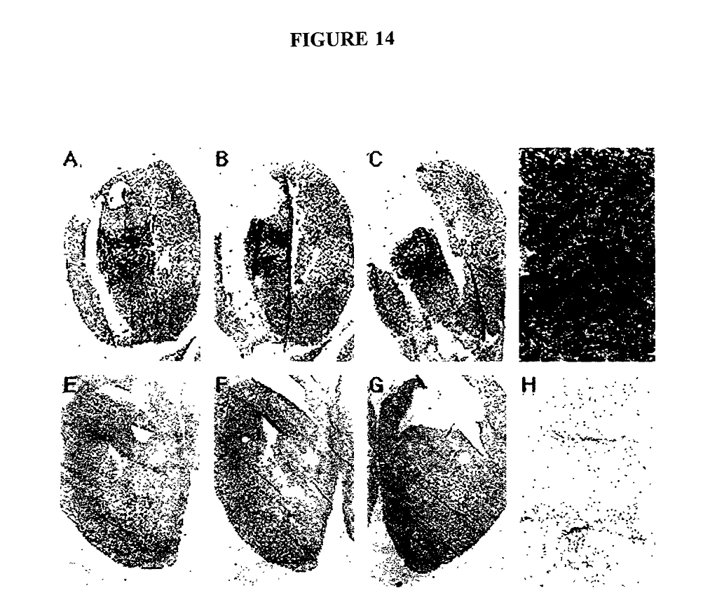

Because Cre-mediated recombination of the co-injected reporter gene could, in theory, be contingent on the episomal state of the loxP-tagged viral genome, additional experiments were conducted. In these experiments, the CMV-driven Cre virus was injected into apical myocardium of adult (9.5 week) CAG-CATZ transgenic mice. Two mice were injected with the Cre virus and two with the empty CMV control; the hearts were harvested at 72 hr for analysis by X-gal staining. Activation of the Cre-dependent lacZ gene was readily detected in the adult ventricular myocytes as shown in FIG. 14, establishing the feasibility for Cre-mediated recombination in this post-mitotic cell background. FIG. 14 shows adult mouse myocardia that were injected with adenovirus bearing CMV-Cre (Panels A-D); or the empty CMV control (Panels E-H), as shown, and were harvested three days after injection. Tissue was embedded in OCT solution (Triangle Biomedical Sciences), and frozen in isopentane over liquid N₂; cryostat sections were prepared using a thickness of 6 mm. X-gal staining of cryostat sections was performed as described in Example 8, except fixation was performed for 15 min at 0°C., and staining was carried out for 12 hr at 37°C. Sections were counterstained with nuclear fast red. Representative low- and high-power fields are illustrated in FIG. 14. As shown, up to 80% of myocytes showed evidence of recombination in the region injected with Ad.CMV.Cre, while no positive cells were detected in mice that received the control virus. Both mice that received the Cre virus expressed LacZ in the area injected, in eight or more sections spaced 250 mm apart. The percentage of LacZ⁺cells ranged from 60-80% at the site injected and, thus, was at least equal to that achieved with Ad/CMV/nls-lacZ. Ten 40× fields, totaling at least 2000 cells, were scored for each mouse. No LacZ staining was detected in control animals injected with the empty virus. Thus, the efficacy for acutely induced recombination in post-mitotic ventricular myocytes after local delivery of Cre approaches the degree of recombination achieved (i.e., more widely throughout the myocardium) by sustained expression of the αMHC-Cre gene.

### EXAMPLE 7

### Deletion of the Rb Gene In Vivo

In this experiment, deletion of a native gene, in vivo, was investigated to further demonstrate the methods of the present invention. Specifically, a key exon from the retinoblastoma gene (Rb) was deleted from mice in vivo. A functional retinoblastoma gene is required for normal murine development. For example, homozygous knockout Rb-1 mice fail to reach term and show a number of abnormalities in neural and hematopoietic development (Clarke et al., Nature 359:328 [1992]). Thus, the ability to target Rb in adult mice in a tissue-specific manner is extremely valuable in developing Rb knock-out animals for study of loss-of-function in particular tissues. For example, the methods of the present invention allow for the generation of Rb knockout mice to generate animal models for characterizing and developing drugs against human retinoblastomas, some of which occur naturally as hereditary Rb loss-of-function mutations.

LoxP-tagged Rb mice (obtained from Anton Berns, Netherlands Cancer Institute) were crossed with αMHC-Cre mice, as described above, to generate animals with a deletion of exon 19 in the Rb gene (i.e., Rb loss-of-function animals). These animals were analyzed for the expression of Rb gene with or without the exon 19 deletion. Genomic DNA was collected from kidney, lung, liver, and heart tissues and analyzed by PCR as described above.

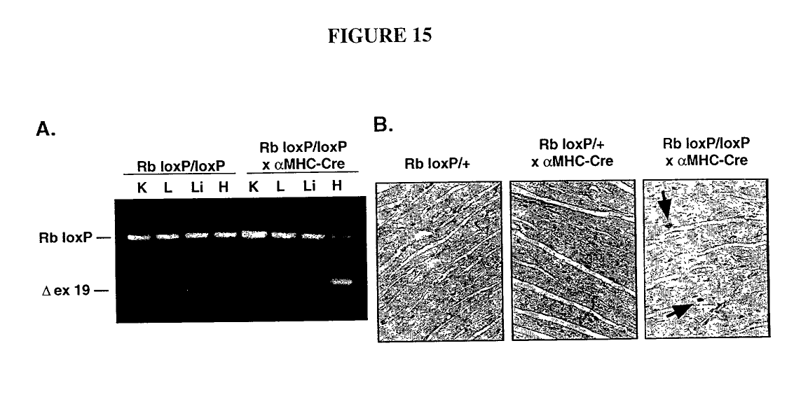

FIG. 15A shows PCR results for the loxP flanked gene (upper band) versus the same gene after Cre-mediated recombination (lower band) in multiple tissues (K=kidney; L=lung; Li=liver; and H=heart). As shown in FIG. 15, recombination was observed only in the heart, and was contingent on inheritance of both the loxP-tagged gene and the αMHC-Cre transgene. FIG. 15B shows that this results in an increased ability of heart muscle to synthesize DNA, demonstrated by immunostaining for the DNA precursor 5′-bromodeoxyuridine. These results further substantiate the utility of the present invention to delete an endogenous gene from the heart, and to alter cardiac growth as the consequence.

### EXAMPLE 8

### Histochemistry

The present example describes some of the histochemical assays utilized during the development of the present invention.

For histological detection of reporter gene expression after injection of recombinant viruses, animal hearts were harvested, bisected, mounted in freezing medium, and frozen in liquid nitrogen, with exceptions where noted. Sets of 4-6 mm cryostat sections were obtained, spaced at 250 μm intervals, using a coronal plane of section, and were fixed in 4% formaldehyde for 10 min. At each interval, one section was used for hematoxylin-eosin staining and one for X-Gal staining using standard methods (See e.g., Ausubel et al., Current Protocols in Molecular Biology, John Wiley & Sons, Inc., New York [1994]), counterstained with nuclear fast red. The percentage of X-Gal-positive cells was estimated for each section, using cell number from the adjacent hematoxylin-eosin-stained section as the denominator.

For luciferase immunocytochemistry, tissue sections fixed as above were incubated with 0.3% H₂O in methanol to inactivate endogenous peroxidase activity, then were incubated with the anti-luciferase antibody at a dilution of 1:75 in phosphate-buffered saline plus 1% bovine serum albumin. Sections were incubated overnight in a humidifying chamber at room temperature and washed 3 times. Bound primary antibody was visualized using biotinylated goat antibody against rabbit IgG, followed by avidin conjugated to horseradish peroxidase (Santa Cruz). Sections were incubated in diaminobenzidine:10%hydrogen peroxide (Pierce) for 10 min and counterstained with eosin.

To detect Cre-dependent β-galactosidase expression by whole-organ staining, tissues were harvested from 6 week-old animals and fixed for 2 hr at room temperature in 2% formaldehyde, 0.2% gluteraldehyde, 0.2% Nonidet P-40, 5 mM ethylenebis(oxyethylenenitrilo)tetraacetic acid, 2 mM MgCl₂, 0.1 M sodium phosphate (pH 7.3). Samples were rinsed for 3×30 min at room temperature in 0.1% deoxycholate, 0.2% Nonidet P-40, 2 mM MgCl₂, 0.1 sodium phosphate (pH 7.3), stained for 3 hr at room temperature with 1 mg/ml 5-bromo-4-chloro-3-indolyl-β-D galactopyranoside (X-gal; Boehringer Mannheim), 5 mM K₄Fe(CN)₆, 5 mM K₃Fe(CN)₆, 0.1% deoxycholate, 0.2% Nonidet P-40, 2 mM MgCl₂, 0.1 sodium phosphate (pH 7.3), and rinsed twice with phosphate-buffered saline as described (Hogan et al., Manipulating the Mouse Embryo: A Laboratory Manual, Cold Spring Harbor Laboratory, Cold Spring Harbor, N.Y., pp 497 [1994]).

### EXAMPLE 9

### Optimization

Like other viral delivery methods, the methods of the present invention may produce some problems with host immune responses (Leiden, N. Engl. J. Med. 333:871 [1995]). The immunogenicity of Cre itself, a bacteriophage protein, may benefit from optimization steps, such as induction of immune tolerance (See e.g., Gilgenkrantz et al., Hum. Gene Ther. 6:1265 [1995]), immunodeficient recipients (Yan and Wilson, Science 273:1862 [1996]), or immunosuppression (Fisher et al., Virology 217:11 [1996]; Kass-Eisler et al., Gene Ther. 1:395 [1994]; Yang et al., J. Virol. 70:6370 [1996]; and Yang et al., Nat. Med. 1:890 [1995]).

Modifications by which the efficiency of the present system might be further improved include employing paired loxP sites that differ from each other to further decrease the theoretical incidence of spontaneous recombination, and improve the ratio of Cre-dependent to Cre-independent target gene expression using this binary switch. However, even in the absence of such modification, the induction resulting from addition of exogenous recombinase compares favorably with the inducibility of other binary systems for regulated gene expression both in vitro and in vivo.

Potential advantages of the αMHC-Cre transgene include the dependence of this promoter on thyroid hormone, suggesting one means to control the timing of recombination. Although this αMHC promoter has the capacity to drive transgenes with exceptional uniformity (Soonpaa et al., Science 264:98 [1994]), strain-to-strain differences in mosaicism were found to occur, which could prove useful in the analysis of dose-dependent defects. Whereas high degrees of uniformity would be especially desirable for mutations of secreted proteins, defined degrees of mosaicism could prove advantageous in contexts including, but not necessarily limited to, cell-autonomous defects (i.e., as foreseen for growth factor receptors, intracellular signaling proteins, structural proteins, enzymes, and transcription factors). As reported recently (Bronson et al., Proc. Natl. Acad. Sci. 93:9067 [1996]), the use of single-copy transgenic mice with integration at a predetermined site provides a plausible means to override the heterogeneity inherent to conventional transgenic methods. Furthermore, as desired, additional enhancers and/or promoters can be used with the present invention to provide the desired tissue specificity, expression level, and inducibility, as described.

In these embodiments, transgenic mouse technology alone, or transgenic mouse technology combined with viral gene transfer, were used to achieve Cre-mediated recombination in cardiac muscle. In vitro, Cre driven by cardiac-specific α-myosin heavy chain (αMHC) sequences elicited recombination selectively at loxP sites in purified cardiac myocytes, but not cardiac fibroblasts. In vivo, this αMHC-Cre transgene elicited recombination in cardiac muscle, but not other organs, as ascertained by PCR analysis and localization of a recombination-dependent reporter protein. Adenoviral delivery of Cre in vivo provoked recombination in post-mitotic, adult ventricular myocytes. Recombination between loxP sites was not detected in the absence of Cre. The methods demonstrated here to evoke gene recombination selectively in myocardium, even in terminally differentiated, post-mitotic ventricular muscle cells, provide means for cardiac drug testing, drug discovery, and generation and characterization of cardiac disease models. However, it is not intended that the present invention be limited to these specific embodiments.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

What is claimed is:

1. A method for gene recombination in post-mitotic cells, comprising:
a) providing:
i) a gene transfer system comprising a DNA sequence encoding a Cre recombinase; and
ii) post-mitotic target tissue comprising target nucleic acid, said target nucleic acid comprising one or more site-specific recombination target sequences; and
b) introducing said gene transfer system to said target tissue,
whereby recombination occurs at said one or more site-specific recombination target sequences.

2. The method of claim 1, wherein said DNA sequence encoding a Cre recombinase further comprises a tissue-specific promoter sequence.

3. The method of claim 1, wherein said gene transfer system comprises a viral gene transfer system.

4. The method of claim 3, wherein said viral gene transfer system comprises an adenoviral gene transfer system.

5. The method of claim 1, wherein said post-mitotic target tissue comprises cardiac tissue.

6. The method of claim 1, wherein said target nucleic acid comprises a gene.

7. The method of claim 1, wherein said one or more site-specific recombination target sequences comprises one or more loxP target sequences.

8. The method of claim 1, wherein said introducing said gene transfer system to said target tissue, comprises injecting said gene transfer system into said target tissue.

9. A method for gene recombination in cardiac tissue, comprising:
a) providing:
i) a viral gene transfer system comprising a DNA sequence encoding a Cre recombinase; and
ii) cardiac tissue comprising target nucleic acid, said target nucleic acid comprising one or more site-specific recombination target sequences; and
b) introducing said viral gene transfer system to said cardiac tissue,
whereby recombination occurs at said one or more site-specific recombination target sequences.

10. The method of claim 9, wherein said cardiac tissue comprises post-mitotic cardiac tissue.

11. The method of claim 9, wherein said DNA sequence encoding a Cre recombinase further comprises a cardiac-specific promoter sequence.

12. The method of claim 9, wherein said viral gene transfer system comprises an adenoviral gene transfer system.

13. The method of claim 9, wherein said target nucleic acid comprises a gene.

14. The method of claim 9, wherein said one or more site-specific recombination target sequences comprises one or more loxP target sequences.

15. The method of claim 9, wherein said introducing said viral gene transfer system to said cardiac tissue, comprises injecting said viral gene transfer system into said cardiac tissue.

16. A method for cardiac-restricted gene recombination, comprising:
a) providing:
i) a viral gene transfer system comprising a DNA sequence encoding a Cre recombinase and a cardiac-specific promoter sequence; and
ii) post-mitotic cardiac tissue comprising target nucleic acid, said target nucleic acid comprising one or more site-specific recombination target sequences; and
b) introducing said viral gene transfer system to said post-mitotic cardiac tissue, whereby recombination occurs at said one or more site-specific recombination target sequences.

17. The method of claim 16, wherein said viral gene transfer system comprises an adenoviral gene transfer system.

18. The method of claim 16, wherein said target nucleic acid comprises a gene.

19. The method of claim 16, wherein said one or more site-specific recombination target sequences comprises one or more loxP target sequences.

20. The method of claim 16, wherein said introducing said viral gene transfer system to said post-mitotic cardiac tissue, comprises injecting said viral gene transfer system into said post-mitotic cardiac tissue.

21. The method of claim 16, wherein said cardiac-specific promoter sequence comprises an a-myosin heavy chain promoter sequence.

22. A non-human mammal, wherein one or more tissues of said non-human mammal comprise tissue prepared according to the method of claim 1.

23. The non-human mammal of claim 22, wherein said non-human mammal is selected from the order Rodentia.

24. The non-human mammal of claim 23, wherein said non-human mammal is selected from the group consisting of mice and rats.

25. A non-human mammal having post-mitotic tissue comprising an altered genotype as compared to wild-type post-mitotic tissue, wherein said altered genotype is the result of tissue-specific recombination.

26. The non-human mammal of claim 25, wherein said post-mitotic tissue comprises cardiac tissue.

27. The non-human mammal of claim 25, wherein said altered genotype comprises a gene knockout.

28. A method for screening compounds for their effect on a transgenic animal, comprising:
a) providing:
i) a transgenic animal, wherein said transgenic animal is the non-human mammal of claim 22; and
ii) a composition comprising a test compound in a form suitable for administration to said non-human mammal; and
b) administering said test compound to said non-human mammal.

29. The method of claim 28, further comprising the step of c) detecting a response of said non-human mammal to said test compound.
